(19) United States
(12) Patent Application Publication
(10) Pub. No.: US 2009/0233858 A1
(43) Pub. Date: Sep. 17, 2009

(54) STRUCTURE OF A PROTEIN PHOSPHATASE 2A HOLOENZYME: INSIGHTS INTO TAU DEPHOSPHORYLATION

(75) Inventors: Yigong Shi, Plainsboro, NJ (US); Yu Chen, Princeton, PA (US); Yanhui Xu, Shanghai (CN)

Correspondence Address: PEPPER HAMILTON LLP, ONE MELLON CENTER, 50TH FLOOR, 500 GRANT STREET, PITTSBURGH, PA, 15219, US

(73) Assignee: THE TRUSTEES OF PRINCETON UNIVERSITY, Princeton, NJ (US)

(21) Appl. No.: 12/393,168

(22) Filed: Feb. 26, 2009

Related U.S. Application Data
(60) Provisional application No. 61/067,227, filed on Feb. 26, 2008.

(51) Int. Cl. A61K 38/16; C12N 9/16; G06G 7/48; G01N 33/53; C12Q 1/34; C07K 14/00; C07K 2/00; C07H 21/00; A61P 43/00; G06F 19/00

(52) U.S. Cl. 514/12; 435/196; 703/11; 435/7.1; 435/18; 530/350; 530/300; 536/23.1; 702/19

(57) ABSTRACT

Embodiments of the present invention relate to crystals and atomic coordinates for PP2A, as well as methods for using these atomic coordinates to prepare modulators of PP2A and inhibitors prepared using such methods. Further embodiments relate to biochemical analyses of the interactions of PP2A alone or in complex with Tau. Further embodiments relate to compositions including mimetics and small molecules, optionally, secondary agents, which may be used to treat disorders in which PP2A activity and/or Tau plays a contributing role.

### CROSS-REFERENCE TO RELATED APPLICATIONS

This application claims priority to and benefit of U.S. Provisional Application No. 61/067,227 entitled “Structure Of A Protein Phosphatase 2a Holoenzyme: Insights Into Tau Dephosphorylation”, filed on Feb. 26, 2008; the entire contents of which are hereby incorporated by reference in its entirety.

### GOVERNMENT INTERESTS

This invention was made with U.S. Government support under Grant No. 5 R01 CA123155 awarded by the National Institutes of Health. The government has certain rights in the invention.

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

Not applicable

2. Description of Related Art

Not applicable

### BRIEF SUMMARY OF THE INVENTION

In some embodiments, the present invention provides compositions comprising a crystal of a PP2A holoenzyme, wherein the holoenzyme comprises an A subunit, a catalytic subunit (C), and a regulatory (B) subunit.

In some embodiments, the present invention provides methods for preparing a PP2A holoenzyme modulating compound comprising applying a three-dimensional molecular modeling algorithm to the atomic coordinates of at least a portion of the PP2A holoenzyme; determining spatial coordinates of the at least a portion of the PP2A holoenzyme; electronically screening stored spatial coordinates of candidate compounds against the spatial coordinates of the at least a portion of the PP2A holoenzyme; identifying a compound that is substantially similar to the at least a portion of the PP2A holoenzyme; and synthesizing the identified compound, wherein the PP2A holoenzyme comprises an A subunit, a catalytic subunit (C), and a regulatory (B) subunit. In some embodiments, the identified compound interrupts the interface between A subunit and the B subunit and/or the identified compound interrupts the interface between the B subunit and Tau.

In some embodiments, the present invention provides pharmaceutical compositions comprising an effective amount of a compound having a three-dimensional structure corresponding to atomic coordinates of at least a portion of a PP2A holoenzyme, wherein said holoenzyme comprises an A subunit, a catalytic subunit (C), and a regulatory (B) subunit and a pharmaceutically acceptable excipient or carrier.

In some embodiments, the present invention provides systems for identifying PP2A modulators comprising: a processor; and a processor readable storage medium in communication with the processor readable storage medium comprising the atomic coordinates of at least a portion of a PP2A holoenzyme, wherein said holoenzyme comprises an A subunit, a catalytic subunit (C), and a regulatory (B) subunit.

In some embodiments, the present invention provides PP2A holoenzyme binding compounds comprising a molecule having a three-dimensional structure corresponding to atomic coordinates derived from at least a portion of an atomic model of the PP2A holoenzyme, wherein said holoenzyme comprises an A subunit, a catalytic subunit (C), and a regulatory (B) subunit.

In some embodiments, the present invention provides recombinant polypeptides comprising a PP2A binding fragment of Tau and/or an isolated nucleic acid encoding a polypeptide comprising a PP2A binding fragment of Tau.

### DESCRIPTION OF DRAWINGS

For a fuller understanding of the nature and advantages of the present invention, reference should be made to the following detailed description taken in connection with the accompanying drawings. The file of this patent contains at least one drawing/photograph executed in color. Copies of this patent with color drawing(s)/photograph(s) will be provided to the USPTO upon request and payment of the necessary fee. All figures where structural representations are shown were prepared using MOLSCRIPT (Kraulis (1991) J Appl Crystallogr 24:946-950) and GRASP (Nicholls et al. (1991) Proteins: Struct Funct Genet 11:281-296).

FIG. 1. Overall structure of the heterotrimeric PP2A holoenzyme involving the Bα subunit. (A) Overall structure of the PP2A holoenzyme involving the Bα subunit and bound to MCLR. The scaffold (Aα), catalytic (Cα), and regulatory B (Bα) subunits are shown in yellow, green, and blue, respectively. MCLR is shown in magenta. Bα primarily interacts with Aα through an extensive interface. Cα interacts with Aα as described (Xing et al., 2006). Two views are shown here to reveal the essential features of the holoenzyme. (B) The regulatory Bα subunit contains a highly acidic top face and a hairpin arm. Bα is in surface representation. Aα and Cα are shown in backbone worm. (C) Comparison of the distinct conformations of the A subunit in the PP2A core enzyme and in the two holoenzymes. FIGS. 1B, 2C, and 4E were prepared using GRASP (Nicholls et al., 1991); all other structural figures were made using MOLSCRIPT (Kraulis, 1991).

FIG. 2. Structural feature of the regulatory B subunit. (A) Sequence alignment of the four isoforms of the regulatory B subunits from humans. Secondary structural elements are indicated above the sequences. Conserved residues are highlighted in yellow. Residues that H-bond to Aα using side chain and main chain groups are identified with red and green circles, respectively, below the sequences. Amino acids that make van der Waals interactions are indicated by blue squares. The sequences shown include all four isoforms of B subunit from humans: alpha (GI: 4506019), beta (GI: 4758954), gamma (GI: 21432089), delta 1 (GI: 51093851) and delta 2 (GI: 51093853). (B) Structure of the B subunit. The β-propeller core is shown in blue; the additional secondary structural elements above the top face are shown in yellow; and the β2C-β2D hairpin arm is highlighted in magenta. Two perpendicular views are shown. (C) The putative substratebinding groove on the top face of the Bα propeller is located in close proximity to the active site of the C subunit of PP2A.

FIG. 3. Specific recognition of the B subunit for the PP2A scaffold subunit. (A) A stereo view of the atomic interactions between the β2C-β2D hairpin arm of Bα and HEAT repeats 1 and 2 of Aα. This interface is dominated by van der Walls contacts. (B) A stereo view of the recognition between the bottom face of Bα and HEAT repeats 3-7. This interface contains a number of hydrogen bonds, which are represented by red dashed lines. (C) Structural comparison of the PP2A holoenzymes involving the regulatory B/B55/PR55 and B′/B56/PR61 subunits.

FIG. 4. Identification of residues in Bα that are critical for binding to the phosphorylated Tau (pTau). (A) Scheme of the in vitro dephosphorylation assay for pTau. There are five major steps as shown. Representative quality of the unphosphorylated and phosphorylated Tau is shown on SDS-PAGE gels stained by coomassie blue (right panels). (B) The heterotrimeric PP2A holoenzyme involving Bα exhibited an enhanced ability to dephosphorylate pTau compared to the heterodimeric PP2A core enzyme. The PP2A concentrations used in lanes 2-6 are 0.73 nM, 2.2 nM, 6.7 nM, 20 nM, and 60 nM. The quality of PP2A core and holoenzymes are shown in the right panel. (C) PP2A holoenzymes involving seven different mutant Bα subunit. The holoenzymes were visualized on SDS-PAGE by coomassie blue staining. (D) Mutations in the Bα subunit affected PP2A-mediated dephosphorylation of pTau. (E) A close-up view of the amino acids that are implicated in binding to pTau. These amino acids are shown in yellow.

FIG. 5. Identification of peptide fragments in Tau that are critical for binding to Bα. (A) A summary of the binding assays between various Tau fragments and the PP2A holoenzyme involving Bα. Potential phosphorylation sites in Tau are indicated by asterisks. (B) A representative native PAGE gel showing interaction between the full-length Tau and the PP2A holoenzyme involving Bα. The free PP2A holoenzyme involving Bα migrated in two discrete bands (lane 2). This result was confirmed by western blot using antibodies specific for Cα and Bα. Binding of the PP2A holoenzyme by Tau resulted in two slower-migrating species. (C) A representative example of the result from gel filtration chromatography. In this example, the Tau fragment (residues 197-259) was incubated with the PP2A holoenzyme involving Bα and applied to gel filtration. Relevant peak fractions from gel filtration were visualized on SDSPAGE by coomassie blue staining. The apparent co-migration of Tau (197-259) with PP2A indicates interaction. The control (free Tau fragment on gel filtration) is shown in the lower panel. (D) A proposed model of PP2A-mediated dephosphorylation of pTau. In this model, pTau binds to the acidic groove on the top face of the B subunit, which presumably facilities access of the nearby phosphorylated serine and threonine residues to the active site of the C subunit of PP2A. Tau contains at least two binding elements for the B subunit, which likely maximize the efficiency of dephosphorylation by enhanced presentation of phosphoamino acids to PP2A.

### DETAILED DESCRIPTION

It must be noted that, as used herein and in the appended claims, the singular forms “a,” “an,” and “the” include plural reference unless the context clearly dictates otherwise. Unless defined otherwise, all technical and scientific terms used herein, have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

The terms “mimetic,” “peptide mimetic,” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically produced peptides, recombinantly or chemically modified peptides, as well as non-peptide agents, such as small molecule drug mimetics as further described below. Mimetic compounds can have additional characteristics that enhance their therapeutic application, such as increased cell permeability, greater affinity and/or avidity, and prolonged biological half-life.

As used herein, the terms “pharmaceutically acceptable,” “physiologically tolerable,” and grammatical variations thereof, as they refer to compositions, carriers, diluents, and reagents, are used interchangeably and represent that the materials are capable of administration upon a mammal without the production of undesirable physiological effects such as nausea, dizziness, rash, or gastric upset.

“Providing,” when used in conjunction with a therapeutic, means to administer a therapeutic directly into or onto a target tissue, or to administer a therapeutic to a patient whereby the therapeutic positively impacts the tissue to which it is targeted.

As used herein, “subject,” “patient” or “individual” refers to an animal or mammal including, but not limited to, a human, dog, cat, horse, cow, pig, sheep, goat, chicken, monkey, rabbit, rat, or mouse, etc.

As used herein, the term “therapeutic” means an agent utilized to treat, combat, ameliorate, or improve an unwanted condition or disease of a patient. Embodiments of the present invention are directed to promote apoptosis and thus, cell death.

The terms “therapeutically effective amount” or “effective amount,” as used herein, may be used interchangeably and refer to an amount of a therapeutic compound component of the present invention. For example, a therapeutically effective amount of a therapeutic compound is a predetermined amount calculated to achieve the desired effect, i.e., to effectively modulate the activity of protein phosphatase 2A (PP2A) and/or Tau.

“Inhibitor” means a compound which reduces or prevents a particular interaction or reaction. For example, an inhibitor may bind to PP2A C-subunit inactivating the C-subunit and inhibiting the phosphatase activity of PP2A. An inhibitor may also inhibit the interaction between subunits of PP2A. An inhibitor may also inhibit the enzymatic activity of PP2A.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable and formed with inorganic acids, such as hydrochloric acid, hydrobromic acid, sulfuric acid, nitric acid, carbonic acid, phosphoric acid, and the like. Organic acids may be selected from aliphatic, cycloaliphatic, aromatic, araliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid, and the like.

Protein phosphorylation and dephosphorylation are essential to all aspects of biology (Hunter, 1995). Protein phosphatase 2A (PP2A) is an important serine/threonine phosphatase that plays a critical role in cellular physiology including cell cycle, cell proliferation, development, and regulation of multiple signal transduction pathways (Janssens and Goris, 2001; Lechward et al., 2001; Virshup, 2000). PP2A is also an important tumor suppressor protein (Janssens et al., 2005; Mumby, 2007). Mutations, total absence or substantial reduction of the scaffold subunit had been linked to a variety of primary human tumors (Calin et al., 2000; Colella et al., 2001; Ruediger et al., 2001; Suzuki and Takahashi, 2003; Takagi et al., 2000; Wang et al., 1998). In addition, truncation of a specific regulatory subunit of PP2A was found to be associated with a highly metastatic state of melanoma cells (Ito et al., 2000; Ito et al., 2003; Koma et al., 2004); gain- and loss-of-function experiments vindicated this regulatory subunit as a tumor suppressor (Chen et al., 2004).

The PP2A core enzyme comprises a 65-kD scaffold subunit (known as A or PR65 subunit) and a 36-kD catalytic subunit (or C subunit). To gain full activity towards specific substrates, the PP2A core enzyme interacts with a variable regulatory subunit to form a heterotrimeric holoenzyme. The variable regulatory subunits are divided into 4 families: B (also known as B55 or PR55), B′ (B56 or PR61), B″ (PR48/PR72/PR130), and B′′ (PR93/PR110), with at least 16 members in these families (Janssens and Goris, 2001; Lechward et al., 2001). In mammalian cells, the A and the C subunits each have two isoforms α and β, which share high sequence similarity (Arino et al., 1988; Green et al., 1987; Hemmings et al., 1990; Stone et al., 1987). In contrast, there is no detectable sequence homology among the four families of regulatory subunits; the expression levels of various regulatory subunits are highly diverse depending upon cell types and tissues (Janssens and Goris, 2001; Lechward et al., 2001). In this regard, the regulatory subunits determine the substrate specificity as well as the spatial and temporal functions of PP2A. Elucidation of the structure of the four different families of PP2A holoenzymes is essential to understanding the function and mechanisms of PP2A.

PP2A is particularly abundant in brains, accounting for up to one percent of total cellular protein mass. An important function of PP2A is to dephosphorylate the hyperphosphorylated Tau protein (Bennecib et al., 2000; Goedert et al., 1995; Gong et al., 2000; Kins et al., 2001; Sontag et al., 1996; Sontag et al., 1999), which has a tendency to polymerize into neurofibrillary tangles, a hallmark of Alzheimer's disease (Goedert and Spillantini, 2006). The hyperphosphorylated Tau also sequesters normal Tau protein, whose function is to promote assembly and stabilization of microtubules (Weingarten et al., 1975; Witman et al., 1976), and thus causes damage to the microtubules (Alonso et al., 1994). PP2A-mediated dephosphorylation of Tau appears to be facilitated by the B/B55/PR55 regulatory subunit (Drewes et al., 1993; Gong et al., 1994). How PP2A specifically recognizes and dephosphorylates pTau remains poorly understood.

Structural investigation has revealed significant insights into the function and mechanisms of PP2A. The A subunit contains 15 tandem repeats of a conserved 39-residue sequence known as a HEAT (huntingtin-elongation-A subunit-TOR) motif (Hemmings et al., 1990; Walter et al., 1989). These 15 HEAT repeats are organized into an extended, L-shaped molecule (Groves et al., 1999). The C subunit recognizes one end of the elongated A subunit by interacting with the conserved ridge of HEAT repeats 11-15 (Ruediger et al., 1994; Ruediger et al., 1992; Xing et al., 2006). Structure of a PP2A holoenzyme involving a B′ regulatory subunit revealed that the B′ subunit is structurally similar to the A subunit and interacts with the ridge of HEAT repeats 2-6 (Cho and Xu, 2006; Xu et al., 2006).

PP2A functions by removing phosphate groups from substrate proteins; ultimately, elucidation of the function and mechanism of PP2A depends on improved understanding of specific PP2A-substrate interactions. However, despite the available structural information, there is a serious lack of understanding on how PP2A specifically facilitates dephosphorylation of target proteins. In addition, the available structural information offers little insights into the function and mechanism of PP2A holoenzymes involving the B, B″, or B″′ regulatory subunits, because these regulatory subunits share no sequence homology with the structurally known B′/B56/PR61 subunit. Among these regulatory subunits, B/B55/PR55 is particularly important because of its intimate link to the neurodegenerative diseases.

In the present application the crystal structure of a PP2A holoenzyme, involving the α isoform of the regulatory B/B55/PR55 subunit (Bα) is disclosed. This represents the first piece of structural information on the regulatory B family and reveals how the B regulatory subunit associates with the PP2A core enzyme to assemble into a heterotrimeric holoenzyme. Importantly, to examine the mechanism of PP2A-mediated dephosphorylation of Tau, an in vitro Tau dephosphorylation assay was reconstituted using recombinant, homogeneous proteins. Using this assay, the Tau-binding element was mapped on the B subunit, identified the Tau peptide motifs that bind to the B subunit, and have disclosed a model for how PP2A holoenzyme facilitates Tau dephosphorylation.

Prior to the present invention, the underlying molecular mechanisms of the interplay between PP2A and its substrates (e.g. Tau) remained largely unknown and there is a long-felt need for a better understanding regarding this interplay and the identification of modulators of the interactions and the regulation of enzymatic activity of PP2A with its substrates

Embodiments of the present invention fulfills these needs and others by better understanding the regulation of PP2A through the elucidation of the crystal structures of the PP2A holoenzyme.

In some embodiments, the polypeptide sequence of PP2A A-subunit comprises SEQ ID NO: 1. In some embodiments, the polypeptide sequences of the catalytic subunit of PP2A, Cα comprises SEQ ID NO: 3. In some embodiments, the polypeptide sequence of the regulatory subunit Bα of PP2A comprises SEQ ID NO: 2.

In some embodiments, the present invention is directed to the atomic coordinates defining the PP2A holoenzyme. In some embodiments, the PP2A holoenzyme comprises an A subunit, a catalytic subunit (C), a regulatory subnit (B), or combinations thereof. The regulatory, B, subunit can be, for example Bα. Embodiments of the present invention are also directed to methods for using the atomic coordinates of the PP2A holoenzyme, mimetics and small molecules prepared using such methods, and pharmaceutical compositions made from mimetics and small molecules so prepared.

In some embodiments, the present invention is directed to a composition comprising a crystal of the PP2A holoenzyme. The PP2A holoenzyme that can form crystals may, for example, comprise an A subunit, a catalytic subunit (C), a regulatory subnit (B), or combinations thereof The regulatory, B, subunit can be, for example Bα. The crystal of the holoenzyme can also comprise microcystin-LR (MCLR). In some embodiments, prior to crystallization the PP2A holoenzyme is incubated with an inhibitor of PP2A. Examples of inhibitors of PP2A include but are not limited to, MCLR, Okadaic Acid, Calyculin A, Cantharidic Acid, Endothall, and Tautomycin. The concentration of the inhibitors of PP2A can vary according to the specificity and the IC₅₀ of each inhibitor. For example, prior to crystallization MCLR can be incubated with the PP2A holoenzyme at a concentration of about 0.5 to about 10 molar equivalence; about 1 to about 5 molar equivalence; about 1 to about 2 molar equivalence; or about 1.2 molar equivalence. The crystals of the holoenzyme can also be generated using selenomethionine-substituted holoenzyme. For example, the proteins that make of the PP2A holoenzyme can be grown in medium where the methionines are replaced with selenomethionines.

In certain embodiments, the PP2A holoenzyme comprises a subunit A that comprises residues 1-589 or 9-0589 of SEQ ID NO: 1. In certain embodiments, the PP2A holoenzyme comprises a B subunit that comprises residues 1-447 or 8-446 of SEQ ID NO: 2. In certain embodiments, the PP2A holoenzyme comprises a C subunit that comprises residues 1-309 or 6-293 of SEQ ID NO: 3.

In various embodiments, the claimed invention relates to methods of preparing crystalline forms of the PP2A holoenzyme by providing an aqueous solution comprising the PP2A holoenzyme that has or has not been incubated with a PP2A inhibitor. A reservoir solution comprising a precipitant may be mixed with a volume of the PP2A holoenzyme and the resultant mixed volume is crystallized. In some embodiments, the crystals may be dissolved and recrystallized. The crystals can be dissolved with the precipitant in a small amount to minimize dilution effects of the other reagents and left to regrow for a period of time.

The proteins can be prepared by any method to isolate purified proteins, such as isolation from E. Coli that overexpress the proteins of interest. The proteins can then be purified to, for example, homogeneity, by gel filtration chromatography. The proteins can also be expressed as fusion proteins or tagged proteins. For example the subunits of the PP2A holoenzyme can be fused with glutathione S transferase (GST) to form a GST fusion protein. The proteins can also be expressed comprising a tag. Examples of tags include, but are not limited to HA, His6, or myc. For example, a subunit of the PP2A holoenzyme can be expressed as His6-tagged full length protein. The proteins can also be expressed in baculovirus-insect cell expression system. For example, baculovirus that encodes for the proteins to be express are used to infect insect cells (e.g. SF9 cells). The infected insect cells then can express the proteins that are encoded by the vectors used to infect the cells. The holoenze can be purified by using the A-subunit to pull out the regulatory and/or catalytic subunits of PP2A. In some embodiments, the catalytic subunit can be methylated. The catalytic subunit can be methylated, for example, by incubating the catalytic subunit with a methyltransferase. An example of a methyltransferase that can methylate the catalytic subunit is, but not limited to, PP2A-specific leucine carboxyl methyltransferase (LCMT). As a source of the methyl group to be transferred to the catalytic subunit S-adenosyl methionine (SAM) can be used. In some embodiments the catalytic subunit in the complex that can be crsystallized or is present is not methylated.

In various embodiments of the method of preparing crystalline forms the PP2A holoenzyme, the concentration of the proteins the aqueous solution may vary, but can be, for example, about 1 to about 50 mg/ml, about 5 to about 15 mg/ml, about 5 to about 10 mg/ml or about 8 mg/ml. Similarly, precipitants used in the invention may vary, and may be selected from any precipitant known in the art. Any concentration of precipitant may be used in the reservoir solution. For example, the concentration can be about 5-10%, about 7-10%. In some embodiments, the concentration is about 7-10% PEG35,000 (w/v). The solutions can also reagents that can assist in obtaining crystals that can defract X-rays to obtain a structure that is at a resolution of at least 5 angstroms or better. An example of additional reagents includes, for example, sodium citrate. The sodium citrate can be present at a concentration of about 0.005 to about 1 M, about 0.005 to about 0.5 M, about 0.005 to about 0.25 M, about 0.005 to about 0.20 M, about 0.005 to about 0.15 M, about 0.1 to about 0.2M, about 0.1 to about 0.15 M. The reservoir solution can also be at various pHs. Examples of pHs that the reservoir solution can be is a pH of about 4 to about 7, about 4 to about 6, about 5 to about 6, or about 5.5.

One skilled in the art will understand that each of these parameters can be varied without undue experimentation and acceptable crystals will still be obtained. In practice, once the appropriate precipitating agents, buffers, or other experimental variables are determined for any given growth method, any of these methods or any other methods can be used to grow the claimed crystals. One skilled in the art can determine the variables depending upon one's particular needs. Various methods of crystallization can be used in the claimed invention, including, for example, vapor diffusion, batch, liquid-bridge, or dialysis crystallization. See, e.g. McPherson et al., Preparation and Analysis of Protein Crystals, Glick, ed. (John Wiley & Co., 1982), pp. 82-159; Jancarik et al., J. Appl. Crystallogr., 24: 409-411 (1991).

In vapor diffusion crystallization, a small volume (i.e., a few milliliters) of protein solution is mixed with a solution containing a precipitant. This mixed volume is suspended over a well containing a small amount, i.e. about 1 ml, of precipitant. Vapor diffusion from the drop to the well will result in crystal formation in the drop.

The dialysis method of crystallization utilizes a semipermeable size-exclusion membrane that retains the protein but allows small molecules (i.e. buffers and precipitants) to diffuse in and out. In dialysis, rather than concentrating the protein and the precipitant by evaporation, the precipitant is allowed to slowly diffuse through the membrane and reduce the solubility of the protein while keeping the protein concentration fixed.

The batch methods generally involve the slow addition of a precipitant to an aqueous solution of protein until the solution just becomes turbid; at this point the container can be sealed and left undisturbed for a period of time until crystallization occurs.

The crystal structure can be determined, for example, by molecular replacement. For example, the structure of the PP2A holoenzyme can be determined by molecular replacement using the PP2A core enzyme and, for example, various WD40 repeats. These the core enzyme and the repeats can be used as a model. Calculations can be peformed by any program capable of performing the appropriate calculations. an Example of a program that is suitable is PHASER. Other programs, such as those described herein, can be used to further refine the structure to obtain a structure that has a least a resolution of less than about 5 angstroms, less than about 4 angstroms, or less than about 3 angstroms. The resolution of the structure, in some embodiments, can be about 2.85 angstroms.

An example of a method to prepare crystals of the PP2A holoenzyme is, but is not limited to, hanging-drop vapor-diffusion method. In the hanging-drop vapor-diffusion method the protein may be mixed with an about equal volume of reservoir solution. The reservoir solution can, for example, comprise PEG35,000, sodium citrate, at a pH of about 5.5. In some embodiments, the method comprises allowing crystals to grow for about 1 week.

Once formed the crystals can be equilibrated in a cryoprotectant buffer containing the reservoir buffer. In some embodiments, the cryoprotectant buffer further comprises about 10-30%, about 15 to about 25%, or about 20% glycerol. The crystals can also be flash frozen in, for example, a cold nitrogen stream at −170° C. The data sets to determine the structure can be collected by any suitable means including, but not limited to, at NSLS beamline X29. The method can also comprise any variation as described in the Examples described herein.

In some embodiments, the crystal of the PP2A holoenzyme has a space group of 14, P1 or C2. 3. In some embodiments, the crystal in a space group of P1 has unit cell dimensions, ±2%, of a=124 Å b=141 Å, c=141 Å, α=79° β=64°, γ=64°. The crystal in the space group P1 can, for example, comprise four complexes in each asymmetric unit. In some embodiments, a crystal in a space group of C2 has unit cell dimensions, ±2%, of a=247 Å b=121 Å, c=172 Å, α=90° β=133°, γ=90°. The crystal in the space group C2 can, for example, comprise two complexes in each asymmetric unit. In some embodiments, a crystal in a space group of 14 has unit cell dimensions, ±2%, of a=182 Å b=182 Å, c=124 Å, α=90° β=90°, γ=90°. The crystal in the space group I4 can, for example comprise 1 complex in each asymmetric unit.

Further embodiments of the present invention provide crystals comprising the PP2A holoenzyme with or without a PP2A inhibitor that can diffract for X-ray determination. The crystal can, for example, diffract X-rays for a determination of structure coordinates to a resolution of a value equal to or less than about 5.0, equal to or less than about 4.0, equal to or less than about 3.0, equal to or less than about 2.85 angstroms. The crystals can also, for example, diffract X-rays for a determination of structure coordinates to a resolution of a value equal to 2.85 angstroms.

The present invention can also provide, in some embodiments, a crystal that has the structure that is defined by the coordinates as shown in the Appendix.

In certain embodiments, the crystals comprising a protein, for example, PP2A holoenzyme, can comprise a methionine that is replaced with a selenomethionine.

Embodiments of the present invention provide a composition comprising a crystal of the PP2A holoenzyme with or without a PP2A inhibitor (e.g. MCLR). The formation of a PP2A complex comprising the various subunits as described herein can be formed under conditions that are effective to form the complex.

In some embodiments, to form the complex of the PP2A holoenzyme the proteins (e.g. subunits) can be contacted with one another under conditions effective to form a complex. An example of conditions that are effective to form the complex include, but is not limited to, where the catalytic subunit of PP2A is methylated. PP2A can be methylated by any enzyme including, but not limited to, PP2A-specific leucine carboxyl methyltransferase (LCMT1). LCMT1 and PP2A can be incubated in the presence of S=adenosyl methionine (SAM) to facilitate methylation.

In embodiments of the present invention, the compositions can also comprise a crystal of the PP2A holoenzyme comprising the properties described in Table 1. In some embodiments, the crystal comprising the complex of the subunits of PP2A comprises a complex wherein the B subunit binds (i.e. has contact with) the A-subunit of PP2A.

In embodiments of the present invention, the crystals can be used to generate diffraction data to determine the atomic coordinates of the PP2A holoenzyme. The coordinates can be determined using any known method and the coordinates can be used, for example, to construct an atomic model of the PP2A holoenzyme. For example, atomic coordinates of the PP2A holoenzyme may be determined from crystallographic diffraction data collected using a combination of molecular replacement and single-wavelength anomalous dispersion. The diffraction and structural data described herein include atomic models for the PP2A holoenzyme. The atomic model of the complex of the PP2A holoenzyme can include, for example, a PP2A complex that comprises an A-subunit, a regulatory (B) subunit, a catalytic (C) subunit, or combinations thereof. The A subunit can be a protein comprising SEQ ID NO:1 or as otherwise described herein. For example, the A subunit can comprise residues 9-589 of SEQ ID NO: 1. The B-subunit can be, for example, Bα or as otherwise described herein. For example, the B subunit can comprise SEQ ID NO: 2, residues 1-447 or residues 8-446 of SEQ ID NO: 2. The catalytic subunit can be, for example, Cα or as otherwise described herein. For example, the catalytic subunit can comprise SEQ ID NO: 3, residues 1-309 of SEQ ID NO: 3 or residues 6-293 of SEQ ID NO: 3.

Various embodiments of the invention are directed to the atomic coordinates of the PP2A holoenzyme and the use of these atomic coordinates to design or identify molecules that specifically inhibit or activate PP2A, or inhibit or enhance the binding (e.g. formation of complex) of the subunits of the PP2A holoenzyme. For example, in one embodiment, the atomic coordinates of the PP2A holoenzyme may be used to design and/or screen inhibitor molecules that bind to the PP2A holoenzyme and disrupt or inhibit the binding of the subunits of the PP2A holoenzyme. In another embodiments, the atomic coordinates of the PP2A holoenzyme may be used to design and/or screen inhibitor molecules that bind to A, B, and/or C subunits of PP2A and, for example, inhibit the ability of the A-subunit to bind with the B subunit of PP2A. In further embodiments, the atomic coordinates of the PP2A holoenzyme may be used to design and/or screen molecules that inhibit the flexibility of PP2A subunit A, PP2A subunit B, and/or PP2A subunit C such that PP2A subunit A, PP2A subunit B, and/or PP2A subunit C may not contact each other or a substrate protein cannot be brought into contact with the active site of the C-subunit of PP2A. In still other embodiments, the atomic coordinates of the PP2A holoenzyme may be used to design and/or screen activators of PP2A by, for example, increasing the affinity of the C-subunit for its substrate.

Further embodiments comprise methods of designing and/or screening of molecules that inhibit PP2A activity. Such methods may include inhibiting the activity of PP2A C-subunit and/or inhibiting the ability of the PP2A A-subunit to bind to other components of PP2A core or PP2A holoenzyme. For example, in various embodiments, binding of an inhibitor molecule to the A subunit of PP2A may selectively reduce or eliminate the activity of PP2A by reducing the ability of PP2A to bind to its substrate by, for example, interrupting the binding interface between PP2A and its substrate. For example, the molecule may inhibit the interactions between the subunits of the PP2A holoenzyme. In other embodiments, binding of an inhibitor molecule to PP2A may reduce or eliminate modifications to the A-, B-, or C-subunits, such as, for example, methylation by inhibiting binding or activity of activating methyl transferases. In additional embodiments, the atomic coordinates of the PP2A holoenzyme described herein may be used to design and/or screen molecules that activate PP2A catalytic activity by, for example, modulating the methylation status of PP2A.

Such molecules as those described herein that for example, inhibit or enhance the binding of the subunits of PP2A to one another may be designed or screened using any method known in the art. For example, in certain embodiments, the atomic coordinates of the PP2A holoenzyme may be identified, reconstituted and/or isolated in silico (i.e., using a computer processor, software, and a computer/user interface) and used to design or screen molecules that may fit within the interface wherein subunits of the PP2A holoenzyme interact with one another. For example, molecules can be designed that may fit within the interface where the A subunit and the regulatory subunit interact with one another.

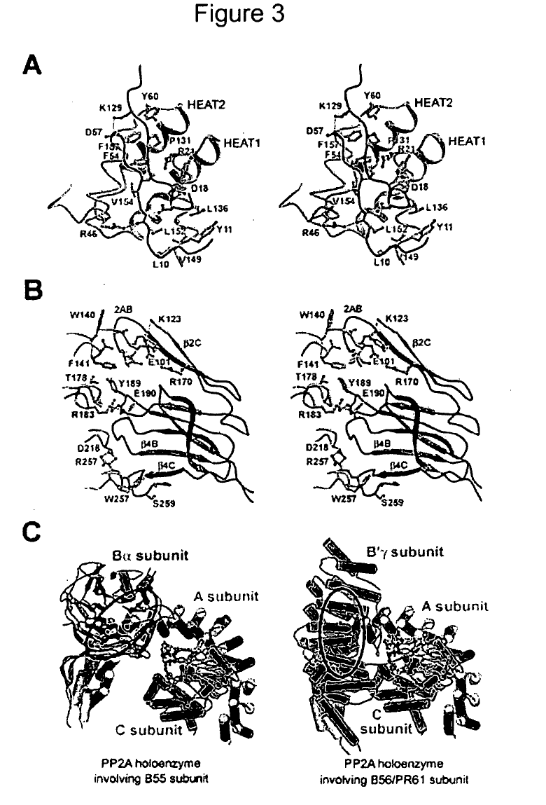

Compounds designed or identified using such methods may substantially mimic the shape, size, and/or charge of a portion of the PP2A holoenzyme. For example, the molecule can mimic the structure formed by the β2C-β2D hairpin arm. In some embodiments, the β2C-β2D hairpin arm comprises residues 125 to 164 of SEQ ID NO: 2. A model of this arm can be made using the coordinates shown in the Appendix. The molecule can, for example, mimic the HEAT repeats in the A subunit that interact with the regulatory subunit. These HEAT repeats can be, for example, HEAT repeats 1 and 2 of the A subunit, which can be seen, for example, in FIG. 3A. In some embodiments, the HEAT repeats 1 and 2 comprises residues 1-80 of SEQ ID NO: 1. The molecule can also mimic the coordinates and the structure formed by the Bα propeller. In some embodiments, the compound mimics the conformation or structure formed by the bottom face of the Bα propeller. In some embodiments, the compound mimics the conformation or structure formed by the ridge of HEAT repeats 3-7 of the A-subunit. In some embodiments, HEAT repeats 3-7 comprise residues 81-274 of SEQ ID NO: 1.

For example, the molecules may mimic the structure formed by the hydrophobic side chains of Pro131 and Phe157 of Bα as indicated by their coordinates in the Appendix. In some embodiments, the molecule may mimic the structure as indicated by the coordinates in the Appendix of residues Phe54 and Tyr60 of subunit A (SEQ ID NO: 1). The molecule may also mimic the structure or surface that is formed by residues Asp57 and Arg21 of the A-subunit SEQ ID NO: 1. The molecule may also mimic the structure or the surface formed by residues Phe54, Tyr60, Asp57, Arg21, or combinations thereof.

The molecule can also mimic the surface or structure according to the coordinates of Arg257 or the resides of loop CD of blade 4 of the Bα subunit. The molecule may also may mimic the structure formed by residue 218 (Asp218) and/or residue 257 (Trp257) of the A-subunit.

As discussed herein Tau can be dephosphorylated by PP2A. Dephosphorylation of Tau is likely an important regulatory mechanism of Tau function. Therefore, in some embodiments, the present invention can be used to identify molecules that can inhibit or enhance the interaction of Tau and PP2A. For example, the a molecule can be created that mimics the surface or structure of PP2A that binds to Tau. The residues that can bind to Tau that can be used as a model for molecule to mimic the structure of can be those that form the central groove on the top face of the β-propeller of the β-subunit. For example, the coordinates of residues 27, 48, 197, and 345 can be used. Additionally, the coordinates fo residues or a portion of the residues present in residues 84-90, 93-95, 178, 179 or combinations thereof may be used to generate a molecule that mimics the structure of these residues. Mutants of these residues can also be used. for example residues can be mutated from E to R, K to e, D to K, or E to A or Y to A, or H to A. Residues can also be mutated to any other residue and then mapped using the coordinates of the holoenzyme (e.g. coordinates described in the Appendix). The residues of the B-subunit of PP2A can also be mutated as described in the Examples section of the present application.

In some embodiments, the surface and/or structure is represented by the coordinates and/or model generated by the coordinates of the residues referred to herein. The coordinates can be those that are shown in the Appendix. Other coordinates can also be used if other coordinates are generated from a crystal of a PP2A holoenzyme. The surface or structures referred to herein may be dependent upon the backbone and/or sidechains of the residues described or referred to.

For example, in one embodiment, a portion of the A-subunit encompassing the atomic coordinates of amino acids 21, 54, 57, 60, 218, 257 or combinations thereof of the A subunit of PP2A (SEQ ID NO: 1) may be used to design and/or screen compounds that substantially mimic the structural features of portions of subunit A of PP2A. In some embodiments, a portion of B-subunit encompassing the atomic coordinates of amino acids 27, 48, 197, and 345, 84-90, 93-95, 178, 179, 131, 157, 257, or combinations thereof may be used to design and/or screen compounds that substantially mimic the structural features of portions of B-subunit and are substantially complementary to the portions that mediate the interaction of B-subunit to the A-subunit of PP2A or Tau. Such compounds may bind to B-subunit, Tau, and/or the A-subunit of PP2A and, for example, inhibit binding of the B-subunit to the A-subunit of PP2A or interrupt interactions between the A-subunit and the B-subunit thereby inhibiting the phosphatase activity of PP2A. Additionally, the compounds may be able to inhibit the interaction between the B-subunit and Tau and then inhibit the dephosphorylation of Tau. In other embodiments, portions of any of the interfaces described and illustrated in any of the figures or coordinates described herein may be used to design and/or screen compounds that may substantially mimic the shape, size, and/or charge of a portion of the PP2A holoenzyme, including but not limited to the portion of PP2A subunits which includes, for example, the interface between the A and B subunits and/or the interaction between the B subunit and Tau.

In some embodiments, a portion of the atomic coordinates defining the B-subunit of PP2A encompassing a binding interface to the A-subunit may be utilized to design and/or screen compounds that may inhibit PP2A activity. For example, a portion of the atomic coordinates of the B-subunit encompassing any of the interfaces described and illustrated in the figures and coordinates described herein may be reconstituted and/or isolated in silico and used to identify compounds that substantially mimic a portion of the B-subunit and/or are substantially complementary to a portion of the interface between B-subunit and the A-subunit. Compounds identified in such embodiments may bind to the B-subunit and inhibit binding of the A-subunit or interrupt interactions at the interface between any or all of the subunits thereby inhibit PP2A enzymatic activity. Compounds that can inhibit the interaction between the B-subunit and Tau can also be identified and made.

In still other embodiments, an inhibitor may be designed or a molecule may screened and identified that inhibits or reduces the flexibility of the A, B, or C-subunits thereby, for example, reducing or eliminating the ability of the subunits to interact with one another, thereby modulating the enzymatic activity of PP2A. Embodiments including the design or screening of inhibitors which reduce flexibility of the subunits of the PP2A holoenzyme may include designing or screening any number of compounds which interact with the C-subunit in any number of ways.

In any of the embodiments described above, a designed or identified inhibitor molecule may have a three-dimensional structure corresponding to at least a portion of the PP2A holoenzyme. For example, an inhibitor may be identified by applying a three-dimensional modeling algorithm to the at least a portion of the atomic coordinates of the PP2A holoenzyme encompassing, for example, a region of the B-subunit where the inhibitor binds or a region of one or more subunits involved in an interface where the subunits make contact with one another or where Tau interacts with the B subunit and electronically screening stored spatial coordinates of candidate compounds against the atomic coordinates of the PP2A holoenzyme or a portion thereof. Candidate compounds that are identified as substantially complementary to the portion of the PP2A holoenzyme modeled, or designed to be substantially complementary to the portion of the PP2A holoenzyme modeled. Candidate compounds so identified may be synthesized using known techniques and then tested for the ability to bind to the PP2A holoenzyme of the subunits themselves. A compound that is found to effectively bind the PP2A holoenzyme may be identified as an “inhibitor” of the PP2A holoenzyme if it can then be shown that the binding of the compound affects the phosphatase activity of PP2A. Such “inhibitors” may then be used to modulate the activity of PP2A in vitro or in vivo. In still other embodiments, such “inhibitors” of PP2A may be administered to a subject or used as part of a pharmaceutical composition to be administered to individuals in need thereof.

The terms “complementary” or “substantially complementary” as used herein, refers to a compound having a size, shape, charge or any combination of these characteristics that allow the compound to substantially fill contours created by applying an three-dimensional modeling algorithm to at least a portion of the PP2A holoenzyme or the entire PP2A holoenzyme. A compound that substantially fills without overlapping portions of the various elements that make up the PP2A holoenzyme, even if various portions of the space remain unfilled, may be considered “substantially complementary”.

The terms “similar” or “substantially similar” may be used to describe a compound having a size, shape, charge or any combination of these characteristics similar to a compound known to bind the PP2A holoenzyme. For example, an identified compound having a similar size, shape, and/or charge to a portion of the C-subunit may be considered “substantially similar” to the C-subunit.

Any inhibitor identified using the techniques described herein, may bind to the PP2A holoenzyme with at least about the same affinity of the protein which binds at a selected interface or a known inhibitor to a known binding site, and in certain embodiments, the inhibitor may have an affinity for the PP2A holoenzyme that is greater than the affinity of the natural or known substrate for the PP2A holoenzyme Thus, such inhibitors may bind to the PP2A holoenzyme and inhibit the activity of PP2A, thereby providing methods and compounds for modulating the activity of PP2A. Without wishing to be bound by theory, modulation of PP2A may reduce PP2A mediated serine/threonine dephosphorylation, and modulating the activity of PP2A may provide the basis for treatment of various cell cycle modulation or proliferative disorders including, for example, cancer and autoimmune disease.

Determination of the atomic coordinates of any portion of the PP2A holoenzyme may be carried out by any method known in the art. For example, the atomic coordinates provided in embodiments of the invention, or the atomic coordinates provided by other PP2A crystallographic or NMR structures including, but not limited to, crystallographic or NMR data for the PP2A holoenzyme, PP2A core, or individual A, B or C components of PP2A, may be provided to a molecular modeling program and the various portions of PP2A holoenzyme described above may be visualized. In other embodiments, two or more sets of atomic coordinates corresponding to various portions of the PP2A holoenzyme may be compared and composite coordinates representing the average of these coordinates may be used to model the structural features of the portion of the PP2A holoenzyme under study. The atomic coordinates used in such embodiments may be derived from purified PP2A holoenzyme, individual A, B or C subunits, or PP2A bound to other regulatory proteins, substrate proteins, accessory proteins, protein fragments or peptides. In general, atomic coordinates defining a three-dimensional structure of a crystal of the PP2A holoenzyme holoenzyme that diffracts X-rays for the determination of atomic coordinates to a resolution of 5 Angstroms or better may be used. In some embodiments, the coordinates used are, for example, those shown in the Appendix.

Having defined the structural features of the PP2A holoenzyme, mimetics or small molecules substantially complementary to various portions of the the PP2A holoenzyme, such as those described above, may be designed. Various methods for molecular design are known in the art, and any of these may be used in embodiments of the invention. For example, in some embodiments, compounds may be specifically designed to fill contours of a portion of the PP2A holoenzyme at the interfaces between the subunits or in portions of the PP2A holoenzyme where other factors or substrate proteins interact. In other embodiments, random compounds may be generated and compared to the spatial coordinates such as a portion of the PP2A holoenzyme. In still other embodiments, stored spatial coordinates of candidate compounds contained within a database may be compared to the spatial coordinates of a portion of the PP2A holoenzyme. In certain embodiments, molecular design may be carried out in combination with molecular modeling.

Methods for performing structural comparisons of atomic coordinates of molecules including those derived from protein crystallography are well known in the art, and any such method may be used in various embodiments to test candidate PP2A binding compounds for the ability to bind a portion of the PP2A holoenzyme. In such embodiments, atomic coordinates of designed, random or stored candidate compounds may be compared against a portion of the PP2A holoenzyme or the atomic coordinates of a compound bound to the PP2A holoenzyme. In other such embodiments, a designed, random or stored candidate compound may be brought into contact with a surface of the PP2A holoenzyme, and simulated hydrogen bonding and/or van der Waals interactions may be used to evaluate or test the ability of the candidate compound to bind the surface of the PP2A holoenzyme. Structural comparisons, such as those described in the preceding embodiments may be carried out using any method, such as, for example, a distance alignment matrix (DALI), Sequential Structure Alignment Program (SSAP), combinatorial extension (CE) or any such structural comparison algorithm. Compounds that appear to mimic a portion of the PP2A holoenzyme under study or a compound known to the PP2A holoenzyme, such as, for example, a substrate protein, or that are substantially complementary and have a likelihood of forming sufficient interactions to bind to the PP2A holoenzyme may be identified as a potential PP2A holoenzyme binding compound.

In some embodiments, compounds identified as described above may conform to a set of predetermined variables. For example, in one embodiment, the atomic coordinates of an identified PP2A holoenzyme binding compound when compared with a PP2A binding compound or a subunit of the PP2A holoenzyme using one or more of the above structural comparison methods may deviate from an rmsd of less than about 10 angstroms. In another embodiment, the atomic coordinates of the compound may deviate from the atomic coordinates of the PP2A holoenzyme by less than about 2 angstroms. In still another embodiment, the identified PP2A holoenzyme binding compound may include one or more specific structural features known to exist in a PP2A holoenzyme binding compound or a subunit of the PP2A holoenzyme, such as, for example, a surface area, shape, charge distribution over the entire compound or a portion of the identified compound.

Compounds identified by the various methods embodied herein may be synthesized by any method known in the art. For example, identified compounds may be synthesized using manual techniques or by automation using in vitro methods such as, various solid state or liquid state synthesis methods. Direct peptide synthesis using solid-phase techniques is well known and utilized in the art (see, e.g., Stewart et al., Solid-Phase Peptide Synthesis, W. H. Freeman Co., San Francisco, Calif. (1969); Merrifield, J. Am. Chem. Soc., 85:2149-2154 (1963)). Automated synthesis may be accomplished, for example, using an Peptide Synthesizer using manufacturer's instructions. Additionally, in some embodiments, one or more portion of the PP2A modulators described herein may be synthesized separately and combined using chemical or enzymatic methods to produce a full length modulator.

Compounds identified using various methods of embodiments of the invention may be further tested for binding to the PP2A holoenzyme and/or to determine the compound's ability to inhibit activity of PP2A or modulate the activity of PP2A by, for example, testing for phosphatase activity or testing the candidate compound for binding to PP2A. Such testing may be carried out by any method. For example, such methods may include contacting a known substrate with an identified compound and detecting binding to PP2A by a change in fluorescence in a marker or by detecting the presence of the bound compound by isolating the PP2A candidate compound complex and testing for the presence of the compound. In other embodiments, PP2A activity may be tested by, for example, isolating a substrate peptide that has or has not been phosphorylated or isolating a PP2A holoenzyme that has been contacted with the candidate compound. Such methods are well known in the art and may be carried out in vitro, in a cell-free assay, or in vivo, in a cell-culture assay.

Embodiments of the invention also include pharmaceutical compositions including inhibitors that bind to PP2A and inhibit PP2A activity or compounds that are identified using methods of embodiments described herein above and a pharmaceutically acceptable carrier or excipient. Such pharmaceutical compositions may be administered to an individual in an effective amount to alleviate conditions associated with PP2A activity.

Various embodiments of the invention also include a system for identifying a PP2A modulator. Such systems may include a processor and a computer readable medium in contact with the processor. The computer readable medium of such embodiments may at least contain the atomic coordinates of the PP2A holoenzyme. In some embodiments, the computer readable medium may further contain one or more programming instructions for comparing at least a portion of the atomic coordinates of the PP2A holoenzyme with atomic coordinates of candidate compounds included in a library of compounds. In other embodiments, the computer readable medium may further contain one or more programming instructions for designing a compound that mimics at least a portion of the PP2A holoenzyme or that is substantially complementary to a portion of the PP2A holoenzyme. In still other embodiments, the computer readable medium may contain one or more programming instructions for identifying candidate compounds or designing a compound that mimics a portion of the PP2A holoenzyme within one or more user defined parameters. For example, in some embodiments, a compound may include a charged molecule at a particular position corresponding to one or more positions within the atomic coordinates of the PP2A holoenzyme, and in other embodiments, the compound may deviate from the carbon backbone or surface model representation of the PP2A holoenzyme by, for example, an rmsd of less than about 10 Å. In still other embodiments, a user may determine the size of a candidate compound or the portion of the PP2A holoenzyme that is utilized in identifying mimetic candidate compounds. Further embodiments may include one or more programming instructions for simulating binding of an identified candidate compound to the PP2A holoenzyme or a portion of the PP2A holoenzyme. Such embodiments may be carried out using any method known in the art, and may provide an additional in silico method for testing identified candidate compounds.

Embodiments of invention described herein may encompasses pharmaceutical compositions comprising a therapeutically effective amount of an inhibitor in dosage form and a pharmaceutically acceptable carrier, wherein the compound inhibits the phosphatase activity of PP2A. In another embodiment, such compositions comprise a therapeutically effective amount of an inhibitor in dosage form and a pharmaceutically acceptable carrier in combination with a chemotherapeutic and/or radiotherapy, wherein the inhibitor inhibits the phosphatase activity of PP2A, promoting apoptosis and enhancing the effectiveness of the chemotherapeutic and/or radiotherapy. In various embodiments of the invention, a therapeutic composition for modulating PP2A activity comprises a therapeutically effective amount of a PP2A inhibitor.

Embodiments of the invention also include methods for treating a patient having a condition characterized by aberrant cell growth, wherein administration of a therapeutically effective amount of a PP2A inhibitor is administered to the patient, and the inhibitor binds to PP2A and modulates cell growth. The method may further include the concurrent administration of a chemotherapeutic agent, such as, but not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds. As used herein, “concurrent administration” may be administration prior to, substantially simultaneous with, simultaneous with or following administration of the PP2A inhibitor.

The PP2A inhibitors of the invention may be administered in an effective amount. In certain embodiments, an “effective amount” is an amount of a preparation that alone, or together with further doses, produces the desired response. This may involve only slowing the progression of the disease temporarily, although it may involve halting the progression of the disease permanently or delaying the onset of or preventing the disease or condition from occurring. This can be monitored by routine methods known and practiced in the art. Generally, doses of active compounds may be from about 0.01 mg/kg per day to about 1000 mg/kg per day, and in some embodiments, the dosage may be from about 50-500 mg/kg. In various embodiments, the compounds of the invention may be administered intravenously, intramuscularly, or intradermally, and in one or several administrations per day. The administration of inhibitors can occur simultaneous with, subsequent to, or prior to chemotherapy or radiation.

In general, routine experimentation in clinical trials will determine specific ranges for optimal therapeutic effect for each therapeutic agent and each administrative protocol and administration to specific patients will be adjusted to within effective and safe ranges depending on the patient's condition and responsiveness to initial administrations. However, the ultimate administration protocol will be regulated according to the judgment of the attending clinician considering such factors as age, condition and size of the patient, the potency of the PP2A inhibitor administered, the duration of the treatment and the severity of the disease being treated. For example, a dosage regimen of a PP2A inhibitor to, for example, reduce cellular proliferation or induce apoptosis can be oral administration of from about 1 mg to about 2000 mg/day, preferably about I to about 1000 mg/day, more preferably about 50 to about 600 mg/day. In certain embodiments, the dosage may be administered once daily or in divided doses, such as in two, three to four divided doses. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient's tolerance permits. Multiple doses per day are contemplated to achieve appropriate systemic levels of compounds. Generally, a maximum dose is used, that is, the highest safe dose according to sound medical judgment. However, an individual patient may insist upon a lower dose or tolerable dose for medical reasons, psychological reasons or for virtually any other reason.

Embodiments of the invention also include a method of treating a patient with cancer or an autoimmune disease by promoting apoptosis, wherein administration of a therapeutically effective amount of one or more PP2A inhibitors, and the PP2A inhibitor inhibits the phosphotase activity of PP2A. The method may further include concurrent administration of a chemotherapeutic agent including, but not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds.

A variety of administration routes are available. The particular mode selected will depend upon the severity of the condition being treated and the dosage required for therapeutic efficacy. The methods of the invention may be practiced using any mode of administration that is medically acceptable, meaning any mode that produces effective levels of active compounds without causing clinically unacceptable adverse effects. Such modes of administration include, but are not limited to, oral, rectal, topical, nasal, intradermal, inhalation, intra-peritoneal, or parenteral routes. The term “parenteral” includes subcutaneous, intravenous, intramuscular, or infusion. Intravenous or intramuscular routes may be particularly suitable for purposes of the present invention.

In one aspect of the invention, a PP2A inhibitor as described herein, with or without additional biological or chemotherapeutic agents or radiotherapy, does not adversely affect normal tissues while sensitizing aberrantly dividing cells to the additional chemotherapeutic/radiation protocols. While not wishing to be bound by theory because the PP2A inhibitors specifically target PP2A, marked and adverse side effects may be minimized. In certain embodiments, the composition or method may be designed to allow sensitization of the cell to chemotherapeutic agents or radiation therapy by administering the ATPase inhibitor prior to chemotherapeutic or radiation therapy.

The term “pharmaceutically-acceptable carrier” as used herein, means one or more compatible solid or liquid fillers, diluents or encapsulating substances which are suitable for administration into a human. The term “carrier” or “excipient” denotes an organic or inorganic ingredient, natural or synthetic, with which the active ingredient is combined to facilitate the application. The components of the pharmaceutical compositions are also capable of being co-mingled with the molecules of the present invention and with each other, in a manner such that there is no interaction which would substantially impair the desired pharmaceutical efficacy.

The delivery systems that may be used in embodiments of the invention are designed to include time-released, delayed release or sustained release delivery systems such that the delivery of the PP2A inhibitors occurs prior to, and with sufficient time, to cause sensitization of the site to be treated. For example, a PP2A inhibitor may be used in conjunction with radiation and/or additional anti-cancer chemical agents. Such systems can avoid repeated administrations of the PP2A inhibitor compound, increasing convenience to the subject and the physician, and may be particularly suitable for certain compositions of the present invention.

Many types of release delivery systems are available and known to those of ordinary skill in the art including, but not limited to, polymer base systems, such as, poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems including, for example: lipids including sterols, such as cholesterol, cholesterol esters and fatty acids or neutral fats, such as mono-, di- and tri-glycerides; hydrogel release systems; sylastic systems; peptide based systems; wax coatings; compressed tablets using conventional binders and excipients; partially fused implants; and the like. Specific examples include, but are not limited to: erosional systems in which the active compound is contained in a form within a matrix such as those described in U.S. Pat. Nos. 4,452,775, 4,667,014, 4,748,034, and 5,239,660 and diffusional systems in which an active component permeates at a controlled rate from a polymer, such as described in U.S. Pat. Nos. 3,832,253, and 3,854,480. In addition, pump-based hardware delivery systems can be used, some of which are adapted for implantation.

In certain embodiments, use of a long-term sustained release implant may be desirable. Long-term release is used herein, and means that the implant is constructed and arranged to deliver therapeutic levels of the active ingredient for at least about 30 days, and preferably about 60 days. Long-term sustained release implants are well-known to those of ordinary skill in the art and include some of the release systems described above.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions may be prepared by uniformly and intimately bringing the active compound into association with a liquid carrier, a finely divided solid carrier, or both and then, if necessary, shaping the product.

Compositions suitable for parenteral administration conveniently include a sterile aqueous preparation of an ATPase inhibitor which is preferably isotonic with the blood of the recipient. This aqueous preparation may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile fixed oils are conventionally employed as a solvent or suspending medium. For this purpose, any bland fixed oil may be employed including synthetic mono- or di-glycerides. In addition, fatty acids, such as oleic acid, may be used in the preparation of injectables. Carrier formulation suitable for oral, subcutaneous, intravenous, intramuscular, etc. administrations can be found, for example, in Remington's Pharmaceutical Sciences, Mack Publishing Co., Easton, Pa. which is incorporated herein in its entirety by reference thereto.

The present invention also provides methods for identifying inhibitors of the interaction between Tau and the B-subunit of PP2A. In some embodiments, B-subunit binding fragments of Tau are used. In some embodiments, these fragments comprise residues 197-259 and/or residues 265-328 of Tau (SEQ ID NO: 4). In some embodiments, a B-subunit binding fragment of Tau is contacted with PP2A and a test compound is introduced to determine if the test compound can inhibit the binding of the Tau fragment to the PP2A holoenzyme. If the Tau fragment is unable to bind or has reduced binding in the presence of the test compound as compared to in the absence of the test compound then the test compound is said to inhibit the binding of Tau to PP2A. In some embodiments, the Tau protein and/or the PP2A holoenzyme or subunits thereof are recombinant proteins are not endogenous proteins isolated from a cell that normally expresses Tau and/or PP2A.

In some embodiments, the compounds identified using the methods described herein can inhibit the dephosphorylation of Tau by PP2A. Any method can be used to determine whether the compound can inhibit the dephosphorylation of Tau. For example, in some embodiments, a PP2A holoenzyme is incubated with Tau or a fragment thereof that can bind to PP2A and the dephoshorylation activity of PP2A as it relates to Tau is measured. A test compound can then be incubated with PP2A and Tau to determine if the test compound inhibits the dephosphorylation of Tau. If the dephosphorylation of Tau is inhibited then the compound is said to be a PP2A inhibitor of Tau dephosphorylation. The compound may inhibit the dephosphorylation either by inhibiting the catalytic activity of PP2A or by inhibiting the binding of PP2A to Tau.

Phosphorlyation status of a protein (e.g. Tau) can be measured by any method known in the art. Methods include, for example, using phospho-specific antibodies that can be used to quantitate the amount of phosphorylated Tau is present. Additional methods include, but are not limited to, using phosphate groups that incorporate ³²P or ³³P and then Tau phosphorylation or the amount that is dephosphorylated can be measured by the amount of the ³²P or ³³P that is incorporated into Tau or released from Tau in the presence of PP2A with or without a test compound. Methods for measuring phosphorylation of a protein are routine and can be modified by one of skill in the art for specific proteins.

The present invention also provides for compositions comprising a PP2A binding fragment of Tau. Such compositions can comprise, for example, residues 197-259 and/or residues 265-328 of Tau. In some embodiments, the compositions comprise a nucleic acid molecule encoding a protein that is a PP2A binding fragment of Tau. In some embodiments, the nucleic acid molecule encodes for residues 197-259 and/or residues 265-328 of Tau. The proteins that can be produced can be recombinant proteins. In some embodiments, the fragment comprises about 60 residues, about 62, about 63, about 64, about 62 to about 125 residues, or about 62 to about 150 residues.

A PP2A binding fragment of Tau is a fragment of Tau that is sufficient to bind to Tau. Fragments of Tau that can bind to PP2A can be identified by, for example, contacting a fragment of Tau with a PP2A holoenzyme and determining whether the fragment binds to PP2A. Methods of determining whether the Tau fragment can bind to PP2A can be any method such as, but not limited to, pull-down assays, IP-Western; GST-fusion pull down assays, and the like. In a GST pull down assay, example, the fragments of Tau are fused with GST and then glutathione beads are used to isolate the Tau fragments. The Tau fragments are then contacted with PP2A to determine if PP2A can bind to the fragment. Methods of determining binding are routine and any such method can be used.

### EXAMPLES

### Example 1

### Assembly and Crystallization of the PP2A Holoenzyme

The human PP2A core enzyme, involving the full-length Aα and Cα, was assembled as previously described (Xing et al., 2006). Human Bα was expressed in baculovirus-infected insect cells and purified to homogeneity. As reported recently (Ikehara et al., 2007), the in vitro assembly of a PP2A holoenzyme between the PP2A core enzyme and the regulatory B subunit does not require carboxyl-methylation of the C subunit (data not shown). The apparent explanation for this observation was revealed by structural and biochemical analysis. Nonetheless, the possibility that the methylated carboxy-terminal residues of the C subunit may play a minor role in the assembled holoenzyme could not be ruled out. Hence we first prepared the fully methylated PP2A core enzyme as described (Xu et al., 2006) and then assembled the heterotrimeric PP2A holoenzyme involving Bα. The methylated PP2A holoenzyme was incubated with 1.2 molar equivalence of microcystin-LR (MCLR) prior to crystallization. After experimenting with over 150,000 crystallization hanging drops, we eventually succeeded in obtaining small crystals of the PP2A holoenzyme. These crystals had poor reproducibility and were sensitive to radiation damage at synchrotron. The structure was determined by molecular replacement, aided by a multi-wavelength anomalous dispersion map. The atomic model has been refined to 2.85 Å resolution (Table 1).

### Overall Structure of the PP2A Holoenzyme

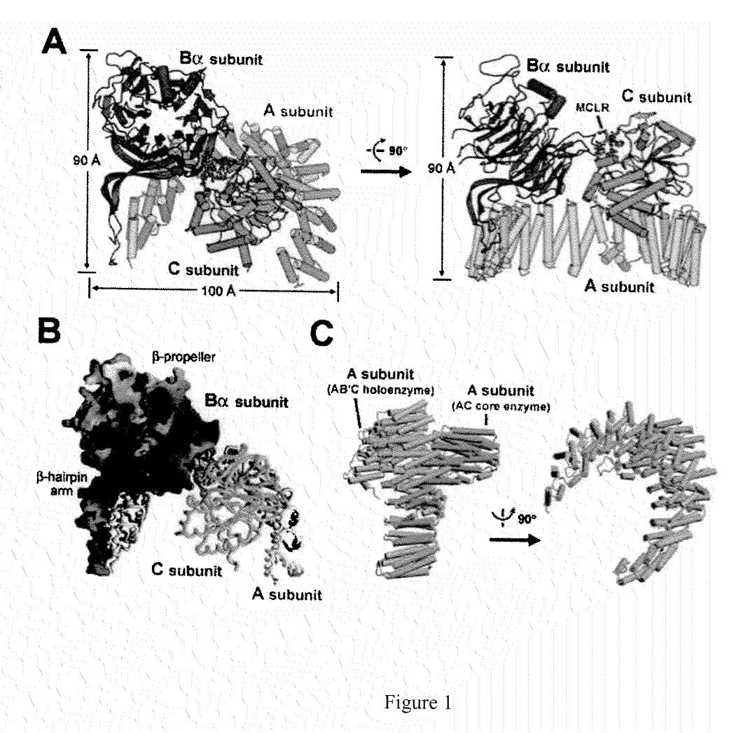

The structure of the 155-kD PP2A holoenzyme exhibits an extended architecture, measuring 100 Å in width, 90 Å in height, and 90 Å in thickness (FIG. 1A, B). There are 15 HEAT repeats in Aα, with each HEAT repeat comprising a pair of antiparallel α helices. Lateral packing among these HEAT repeats gives rise to a horseshoe-shaped structure characterized by double-layered α helices. The loop region connecting two adjacent helices within each HEAT repeat forms a contiguous, conserved ridge (Groves et al., 1999). Compared to the A subunit in the PP2A core enzyme (Xing et al., 2006) or holoenzyme involving the B′ subunit (Cho and Xu, 2006; Xu et al., 2006), Aα displays significant conformational differences (FIG. 1C). As previously observed (Xing et al., 2006), Cα binds to one end of the A subunit through interactions with the ridge of HEAT repeats 11-15.

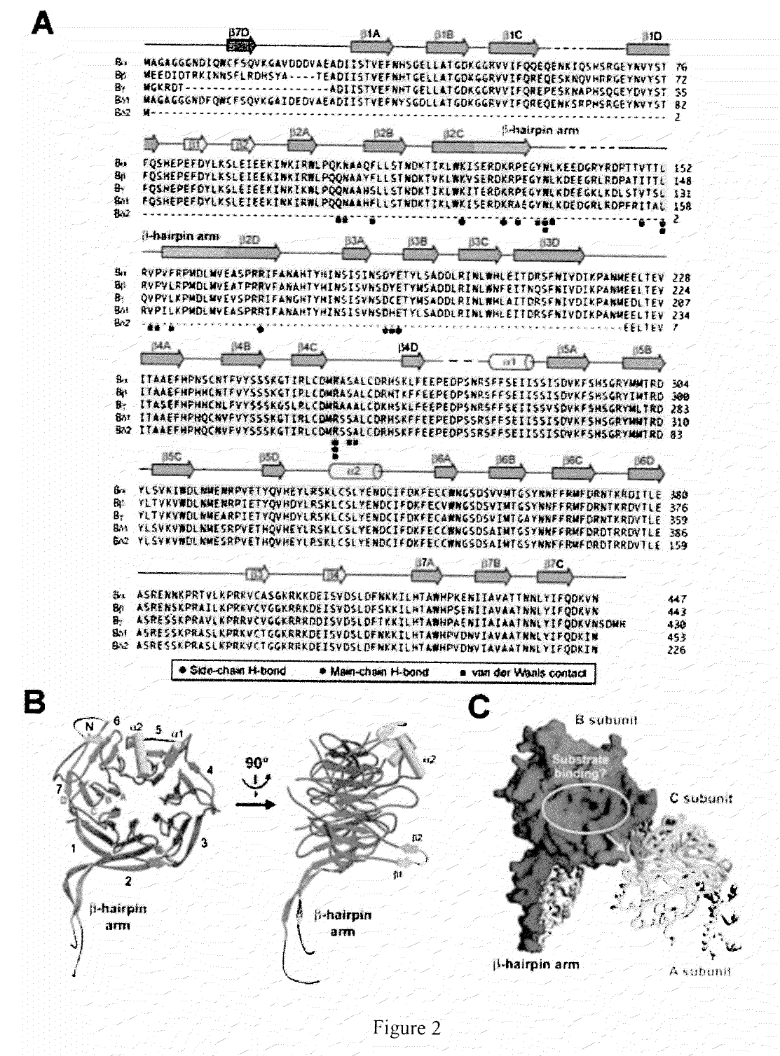

The core of the regulatory Bα subunit forms a 7-bladed β-propeller, with each blade comprising 4 anti-parallel β-strands (FIGS. 1 and 2). By convention of the WD40 domain structure (Wall et al., 1995), the four β-strands in each blade are designated A, B, C, and D, radiating from the center of the torus-like structure. In the middle of the top face of the β-propeller (convention of Wall et al., 1995), there is a highly acidic groove (FIG. 1B). The location and size of the groove are reminiscent of a peptide-binding site that has been observed in other cases (Wilson et al., 2005). In addition to the canonical core structural elements of β-propeller, Bα also contains two β-hairpins and two α-helices, all of which are located above the top face. These additional structural elements contribute to the formation of the putative substrate-binding groove. In blade 2, β-strands C and D extend out of the propeller and form a β-hairpin arm that grabs onto the A subunit as described herein.

Bα makes extensive interactions with the Aα subunit (FIG. 1). The bottom face of the propeller binds to the ridge of HEAT repeats 3-7. The β2C-β2D hairpin arm reaches down to interact with HEAT repeats 1 and 2 (FIGS. 1 and 2). Unlike the PP2A holoenzyme involving the B′ subunit, Bα makes few interactions with the C subunit, with Leu87 from Bα making van der Waals contacts to Val126 and Tyr127 of the C subunit. The methylated carboxy-terminal tail of the C subunit does not have well-defined electron density and appears to be disordered in the crystals. This structural observation is consistent with the biochemical data that methylation is unnecessary for the in vitro assembly of PP2A holoenzyme involving the B family subunits (Ikehara et al., 2007). These observations further suggest that the Bα subunit may form a stable complex with the isolated A subunit. This prediction has been confirmed by our biochemical analysis (data not shown).

### Interface Between the Regulatory and the Scaffold Subunits

The continuous interface between Bα and the A subunit can be described into two portions. One portion is mediated by the β2C-β2D hairpin arm of Bα, which make extensive van der Waals interactions with residues in HEAT repeats 1 and 2 of the A subunit (FIG. 3A). The other portion is dominated by hydrogen bonds (H-bonds), between amino acids in the bottom face of the Bα propeller and the ridge of HEAT repeats 3-7 (FIG. 3B). These interactions result in the burial of 4,270 Å2 exposed surface area.

The hydrophobic amino acids from the β2C-β2D arm of Bα inter-digitate with surrounding residues that are located in the outer-layer of α-helices in HEAT repeats 1 and 2 (FIG. 3A). In particular, the hydrophobic side chains of Pro131 and Tyr157 of Bα make multiple van der Waals contacts to Phe54, Tyr60, and the aliphatic portion of side chains in Asp57 and Arg21. These interactions likely make a major contribution to the binding affinity between Bα and the A subunit. Supporting this analysis, deletion of the β2C-β2D arm in Bα resulted in complete loss of interaction between Bα and the A subunit (data not shown).

The specificity of the interaction appears to be provided by 7 inter-molecular H-bonds at the interface between the bottom face of the Bα propeller and the ridge of HEAT repeats 3-7 (FIG. 3B). In particular, the guanidinium group of Arg257 from loop CD of blade 4 donates a pair of charge-stabilized H-bonds to the side chain carboxylate of Asp218 in the A subunit; these interactions are further buttressed by a main chain H-bond between carbonyl oxygen of Arg257 and amide nitrogen of Trp257.

All amino acids in Bα that H-bond to residues in the A subunit are invariant in the β, γ, and δ1 isoforms of the regulatory B family; whereas the Bα amino acids that make van der Waals contact to the A subunit are conserved (FIG. 2A). This analysis suggests that Bβ, Bγ, and Bδ1 should also interact with the A subunit identically as observed in our crystal structure. Interestingly, however, the Bδ62 subunit contains a large truncation, which results in the removal of blades 1, 2, and 3 (FIG. 2A). Because most PP2A binding elements are contained within blades 2-4, the Bδ2 subunit is likely to have lost its ability to form a PP2A holoenzyme.

### Comparison of Holoenzymes Involving B and B′

Comparison between structures of the holoenzyme involving the regulatory B subunit and that involving the B′ subunit (Cho and Xu, 2006; Xu et al., 2006) revealed interesting functional similarity. In both cases, the regulatory subunit recognizes the amino-terminal HEAT repeats of the A subunit, with Bα interacting with HEAT 1-7 and B′γ binding to HEAT 2-8 (FIG. 3C). In both cases, the putative substrate-binding site is located on the top face of the regulatory subunit, at a position that is proximal to the active site of the C subunit of PP2A. Thus a major function of both regulatory subunits appears to facilitate the targeting of the substrate phosphoprotein to the dephosphatase activity of PP2A.

Important structural differences underlie the contrasting functions of the B and B′ families of regulatory subunits. First, they share no structural similarity, as reflected by their diverging sequences. The B subunit is a 7-bladed β-propeller whereas the B′ subunit comprises 8 HEAT-like repeats. Second, the B′ subunit makes significant interactions with the C subunit of PP2A, which consequently strengthens the inter-subunit packing, making the resulting holoenzyme relatively compact and rigid (FIG. 3C). In contrast, the B subunit makes few interactions with the C subunit and the holoenzyme complex appears to be considerably looser compared to that involving B′.

### In vitro Reconstitution of a Tau Dephosphorylation Assay

Hyperphosphorylation of the Tau protein is thought to be a major contributing factor for formation of the neurofibrillary tangles in the brains of Alzheimer's disease patients (reviewed in (Gong et al., 2005)). Dephosphorylation of the phosphorylated Tau protein (pTau) has been shown to be mediated mainly by the heterotrimeric PP2A holoenzyme involving the B family of regulatory subunits (Bennecib et al., 2000; Drewes et al., 1993; Goedert et al., 1995; Gong et al., 1994; Gong et al., 2000; Kins et al., 2001; Sontag et al., 1996; Sontag et al., 1999). In the past, biochemical investigation of this process relied on PP2A holoenzymes and pTau, both purified from animal tissues. This experimental setup, coupled with the lack of structural information, did not allow mechanistic understanding of PP2A-mediated dephosphorylation of pTau. For example, the endogenous nature of PP2A and pTau did not allow assessment of the roles of candidate amino acids. The advent of the structure of PP2A holoenzyme involving Bα prompted us to reconstitute an in vitro assay for pTau dephosphorylation.

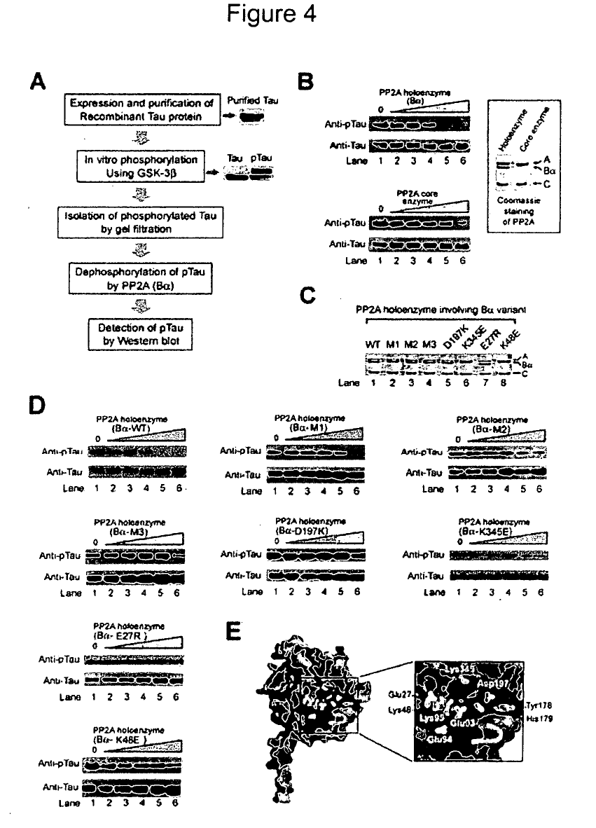

In this assay, all protein components were derived from recombinant expression and in vitro manipulation (FIG. 4A). A major splice variant of human Tau (4R0N), which contains 4 microtubule-binding repeats (Gong et al., 2005), was over-expressed in E. coli and purified to homogeneity using chromatography. The purified Tau was phosphorylated in vitro using the protein kinase GSK-3β and the phosphorylated Tau (pTau) was further purified by gel filtration. Finally, pTau was dephosphorylated by recombinant PP2A and the extent of Dephosphorylation was examined by an antibody that specifically recognizes phosphorylated Ser396 (which corresponds to Ser338 in Tau-4RON).

Using this in vitro assay, the heterotrimeric PP2A holoenzyme involving the Bα subunit efficiently dephosphorylated pTau (FIG. 4B, top panel). The function of Bα and the specificity of this reaction were manifested by the observation that the heterodimeric PP2A core enzyme exhibited a markedly reduced ability to dephosphorylate pTau compared to the holoenzyme (FIG. 4B, bottom panel). In another control experiment, the heterotrimeric PP2A holoenzyme involving the B′γ subunit displayed a further decreased activity compared to the PP2A core enzyme (data not shown), suggesting that the presence of the B′γ subunit may limit access of the pTau substrate to the active site of the C subunit.

### Identification of the Tau-Binding site on B Subunit

Reconstitution of the pTau dephosphorylation assay allowed the identification of Tau-binding site on the B subunit through mutagenesis. Previous studies on β-propeller proteins show that the central groove on the top face of the β-propeller represents a candidate binding site for ligand peptide (Wilson et al., 2005). To examine this scenario, we generated seven baculoviruses, each containing a different Bα mutant for expression in insect cell. Then we individually purified the seven Bα mutants, assembled the corresponding PP2A holoenzymes, each involving a different Bα mutant, and purified these holoenzymes to homogeneity (FIG. 4C). The mutations affect amino acids that are located in or close to the central acidic groove on the top face of the β-propeller. Among the seven mutants, four contain missense mutations (E27R, K48E, D197K, and K345E), each involving changing the charge to the opposite type. The other three are composite mutations: M1 involves replacing seven residues Phe84-Leu90 in the β1β hairpin with two amino acids Gly-Gly; M2 and M3 involve mutating Glu93-Glu94-Lys95 and Tyr178-His179 to Ala93-Ala94-Ala95 and Ala178-Ala179, respectively.

These mutations exhibited different effects on Bα-mediated dephosphorylation of pTau (FIG. 4D). The missense mutant Bα-K345E displayed a similar activity as the WT Bα, suggesting that Lys345 may not be critical for binding to pTau. In contrast, all other Bα mutants showed varying degrees of compromised ability to facilitate the dephosphorylation of pTau. For example, the ability of the PP2A holoenzyme involving Bα-E27R or Bα-D197K to dephosphorylate pTau was even slightly worse than the heterodimeric PP2A core enzyme. These results suggest that the central groove on the top face of the Bα propeller is the likely binding site for Tau and that a cluster of amino acids on one side of the groove may play a critical role in binding to pTau (FIG. 4E).

### Identification of Bα-Binding Sequences in Tau

Next, we sought to identify the Bα-binding sequences in Tau. The primary sequences of all isoforms of Tau contain an unusually high percentage of hydrophilic amino acids and many proline residues. The sequence feature, as well as computer-based sequence analysis, suggested that Tau is unlikely to be a folded protein. Consistent with this analysis, the Tau protein, both derived from bovine brain and recombinant expression in E. coli, was previously shown to contain little or no secondary structure (Cleveland et al., 1977; Wille et al., 1992). We confirmed this conclusion by performing circular dichroism study on the full-length, unphosphorylated splice variant 4R0N of Tau (data not shown). The lack of folded structure in Tau justified the strategy of dividing the full-length Tau proteins into overlapping peptide fragments, which are subsequently evaluated for their ability to interact with the PP2A holoenzyme involving Bα.

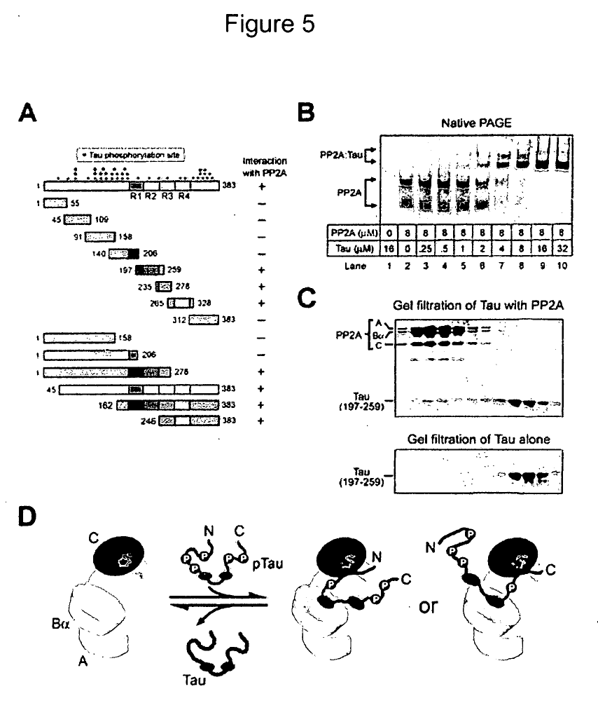

We generated and purified 18 overlapping Tau fragments (FIG. 5A and data not shown). Two different binding assays were used to examine the interaction between each of the 18 Tau fragments and the PP2A holoenzyme involving Bα: polyacrylamide gel electrophoresis (PAGE) under native conditions (FIG. 5B) and gel filtration (FIG. 5C). Because of its sensitive nature for the detection of protein-protein interaction, native PAGE was first employed to assess binding of the various Tau fragments to PP2A. The results were further confirmed by gel filtration chromatography. Our analysis revealed that the full-length Tau binds to the PP2A holoenzyme with an affinity of approximately 3 μM (FIG. 5B). These experiments identified two non-overlapping peptide segments of Tau that are capable of binding to the PP2A holoenzymes: residues 197-259 and residues 265-328 (FIG. 5A). This result suggests that Tau contains at least two PP2A-binding elements. The presence of more than one PP2A-binding site in Tau may greatly facilitate the dephosphorylation of hyperphosphorylated Tau (FIG. 5C), because hyperphosphorylated Tau is thought to contain multiple phosphorylated Ser/Thr residues that are spread throughout the sequences (FIG. 5A).

In the last two years, there has been a rapid accumulation of structural information on PP2A and related proteins, including the PP2A phosphatase activator (Chao et al., 2006; Leulliot et al., 2006; Magnusdottir et al., 2006), the PP2A core enzyme (Xing et al., 2006), the PP2A holoenzyme involving B′ subunit (Cho and Xu, 2006; Xu et al., 2006), PP2A binding protein Tap42/alpha4 (Yang et al., 2007), and the PP2A scaffold subunit bound to small t antigen of SV40 (Chen et al., 2007; Cho et al., 2007). The structural information greatly improved our understanding on some aspects of PP2A assembly, function, and regulation. However, mechanistic understanding of PP2A function and regulation is far from complete. It is fair to say that what we know today represents a very small proportion of what is required to have a comprehensive understanding on the function and mechanisms of PP2A. In particular, there is no structural information on the PP2A holoenzymes involving the B/B55/PR55 or the B″/PR72 families of regulatory subunits. There is a serious lack of structural information on how LCMT1 and PME-1 regulate the reversible methylation of PP2A and how methylation impacts on the assembly of the holoenzymes in vitro. Perhaps more importantly, despite the fact that PP2A functions through dephosphorylation of substrate phosphoproteins, how PP2A recognizes substrate proteins and mediates this activity remain largely unexplored. The major obstacles for solving these problems appear to be technical challenges in dealing with what is now known a very tough protein complex.

In this study, we report two major advances. First, we report the crystal structure of the PP2A holoenzyme involving the B/B55/PR55 family of regulatory subunits. This structure reveals how the Bα subunit specifically recognizes the PP2A core enzyme and how Bα may facilitate substrate dephosphorylation. This structure also represents the first piece of structural information on the B/B55/PR55 family of regulatory subunits, which contains seven WD40 repeats rather than five as previously thought (Janssens and Goris, 2001). Second, we reconstituted a Tau dephosphorylation assay and applied this assay to characterize the interaction between Tau and Bα in the context of PP2A holoenzyme. Our assay relies completely on recombinant components, rather than endogenous materials, and thus allows us to manipulate each component through mutagenesis—strategy required for mechanistic understanding of PP2A function. Using this strategy, we mapped the respective binding epitopes on Bα and on Tau.

Our biochemical characterization suggests that at least two separate peptide fragments of Tau have the ability to interact with the acidic groove of Bα. The presence of more than one PP2A-binding site allows Tau to “slide” on Bα so as to more efficiently present nearby phosphoserine/phosphothreonine residues to the C subunit of PP2A for dephosphorylation. Interestingly, the two putative Bα-binding elements fall within the microtubule-binding repeats of Tau (FIG. 5A). This result is in excellent agreement with a previous study that mapped the Bα-binding element to be within the microtubule-binding region (Sontag et al., 1997). These two Bα-binding repeats are characterized by an enrichment of positively charged amino acids such as lysine and arginine. For example, the Tau fragment 197-259 is highly basic, with 11 lysine residues. This sequence feature agrees well with the acidic nature of the putative substratebinding groove on the Bα subunit. The minimal or consensus peptide that retains binding to the B subunit remains to be identified.

The total reconstitution of Tau dephosphorylation in vitro using homogeneous, recombinant proteins may represent an important step towards deciphering the underpinnings of PP2A-mediated regulation of Tau. GSK-3β, which potently phosphorylates Tau at multiple sites in vitro (reviewed in (Gong et al., 2005)), was used as the kinase for Tau in our assay. Compared to the unphosphorylated Tau, pTau showed retarded mobility on SDS-PAGE gels (FIG. 4). Consistent with published reports, Ser396 was among the Ser/Thr residues in Tau that were phosphorylated by GSK-3β and was recognized by a specific antibody (FIG. 4). The extent of dephosphorylation of pSer396 was used as a direct readout of PP2A activity.

Previous studies suggested that carboxy-methylation of the C subunit was important for the assembly of PP2A holoenzymes involving the B subunits in cells (Bryant et al., 1999; Kloeker et al., 1997; Longin et al., 2007; Tolstykh et al., 2000; Wei et al., 2001; Wu et al., 2000; Yu et al., 2001). A common feature of these studies is that the assembly of PP2A holoenzymes was investigated in cells, rather than in vitro using purified recombinant proteins. In contrast, a recent study using purified proteins showed that the methylation status of the C subunit had no impact on the in vitro assembly of PP2A holoenzyme involving the B subunit (Ikehara et al., 2007). Our structural analysis supports this conclusion. In fact, the carboxy-terminal 14 amino acids of the C subunit are disordered in the crystals and are dispensable for formation of the PP2A holoenzyme in vitro. This conclusion was also confirmed using a carboxy-terminally truncated C subunit, which retained the same binding affinity for the formation of the holoenzyme as that of the full-length, methylated C subunit (data not shown). Similarly, methylation of the C subunit was shown to have little impact on the in vitro assembly of the PP2A holoenzyme involving the B′ subunit (Xu et al., 2006).

These observations argue strongly that the carboxy-methylation of the C subunit is not required for the in vitro assembly of PP2A holoenzymes involving the B and B′ regulatory subunits. If methylation is not required for PP2A holoenzyme assembly in vitro, why does it appear to play an important role in cells (Bryant et al., 1999; Kloeker et al., 1997; Longin et al., 2007; Tolstykh et al., 2000; Wei et al., 2001; Wu et al., 2000; Yu et al., 2001)? One possibility is that the carboxy methylation mainly serves as a signal for assembly of the PP2A holoenzyme. For example, the regulatory subunits may be sequestered in a specific cellular compartment, and the methylated carboxy-terminus of the C subunit may allow its targeting to this location for holoenzyme assembly. Another example is that the methylated carboxy-terminus may help recruit assembly factors that actively promote assembly of the PP2A holoenzymes. Examination of these hypotheses awaits future experiments. Interestingly, a recent cell biological investigation concluded that methylation is not required for the cellular assembly of PP2A holoenzymes involving the B′ and B″ regulatory subunits (Longin et al., 2007).

### Example 2

### Experimental Procedures

### Protein Preparation and Assembly of PP2A Holoenzyme

All constructs and point mutations were generated using a standard PCR-based cloning strategy. Aα (residues 1-589) was overexpressed in E. coli as a fusion protein with glutathione S transferase (GST) and purified as described (Xu et al., 2006). Full-length His6-tagged Cα (residues 1-309) and Bα (residues 1-447) were co-expressed in baculovirus-infected insect cells. The PP2A holoenzyme was purified to homogeneity first by glutathione sepharose 4B resin, using GST-Aα to pull out Bα and Cα, followed by anion exchange and gel filtration chromatography. We also attempted assembly of the holoenzyme by first reconstituting the PP2A core enzyme, which was methylated by a PP2A-specific leucine carboxyl methyltransferase (LCMT) in the presence of S-adenosyl methionine (SAM), and then incubating the homogeneously methylated PP2A core enzyme with the Bα subunit. Both assembly protocols gave rise to identical holoenzymes as examined by phosphatase assays and identical crystals. To facilitate structure determination, we also prepared the PP2A holoenzyme complex using seleno-methionine-substituted Aα, Cα, and Bα proteins using a published protocol (Cronin et al., 2007).

### Crystallization and Data Collection

Diffracting crystals were obtained for the PP2A holoenzyme described above, which was incubated with 1.2 molar equivalence of MCLR prior to crystallization. We also generated crystals of the holoenzyme using selenomethionine-substituted holoenzyme. Crystals were grown by the hanging-drop vapor-diffusion method by mixing the protein (˜8 mg/ml) with an equal volume of reservoir solution containing 7-10% PEG35,000 and 0.1-0.15 M Sodium Citrate pH 5.5. Small crystals appeared within a few days. The crystals were in three closely-related crystal forms: P1 with a=124 Å, b=141 Å, c=141 Å, α=79, β=64, and γ=64 with 4 complexes in the asymmetric unit (AU); C2 with a=247 Å, b=121 Å, c=172 Å, and β=133 with 2 complexes per AU; I4 with a=b=182 Å, c=124 Å with 1 complex per AU. Most of the structural work and the definitive refinement were done with the C2 form. Crystals were slowly equilibrated in a cryoprotectant buffer containing reservoir buffer plus 20% glycerol (v/v) and were flash frozen in a cold nitrogen stream at −170° C. The native and selenium MAD data sets were collected at NSLS beamline X29 and processed using the software Denzo and Scalepack (Otwinowski and Minor, 1997).

### Structure Determination

The structure was determined by molecular replacement using the PP2A core enzyme (Xing et al., 2006) and various WD40 repeats as a model, against an initial 3.5 Å native dataset in the C2 form. Molecular replacement solutions of the P1 and I4 form confirmed the close relationships between these crystal forms. Calculations were performed with the program PHASER (McCoy et al., 2005). Structure determination was complicated by the apparent flexibility of the complexes with the carboxy-terminal end of the Aα subunit and the Cα subunit displaying elevated B-factors. Two AC complexes were assembled based on molecular replacement solutions of the Cα domain and three fragments of the Aα domain. Based on this solution, it was not possible to build the B subunit. A 5.5 Å resolution Ta6Br12 MAD map, calculated using SHELX (Sheldrick, 2008) and SHARP, in the P1 crystal form confirmed the presence of the B subunit and the packing arrangement. In the absence of an available homologous structure for the B subunit an ensemble of five superimposed WD40 domains with trimmed loops was used to find a single B subunit in the P1 crystal form, and the position was confirmed by reference to the Ta6Br12 MAD map. The second B subunit was generated using the known non-crystallographic symmetry relationship. Superimposition of the heterotrimeric complex in the C2 form showed that it was compatible with existing maps and packing in that form.

Despite low homology between the initial model and the B subunit sequence, modelphased 2-fold averaged 2Fo-Fc, alpha-calc maps were sufficient to make modifications to the poly-Ala backbone and successive improvements to the model led to the appearance of interpretable side-chain density. Addition of a 2.9 Å native dataset, and use of the model-phased SeMet anomalous difference map from a 3.8 Å SeMet MAD dataset enabled definitive interpretation of the sequence for the B subunit. The structure was refined at 2.85 Å resolution using the program CNS (Brunger et al., 1998), incorporating non-crystallographic symmetry restraints between the two heterotrimeric complexes. The final atomic model contains amino acids 6-293 for Cα, residues 9-589 for Aα, and residues 8-137 and 146-446 for Bα. There is no electron density for residues 294-309 of Cα, and residues 138-145 of Bα; we presume these regions are disordered in the crystals.

### Methylation of PP2A Core Enzyme by LCMT

This assay was performed as previously described (Xu et al., 2006).

### Native PAGE and Gel Filtration

These assays were performed as previously described (Xing et al., 2006).

### Phosphorylation and Dephosphorylation Assays of Tau Protein

Bacterially expressed Tau was purified by ion-exchange chromatography and gel filtration to homogeneity. The phosphorylation reaction was carried out by mixing purified Tau with GSK3β (Upstate Biotechnology) in the presence of 2 mM ATP and 10 mM MgCl₂ in phosphorylation buffer (8 mM Tris-Cl buffer pH7.5, 0.2 mM EDTA) at 37° C. for 16 hours. Phosphorylated Tau (pTau) was further purified by gel filtration.

In dephosphorylation assay of pTau, 0.36 μM pTau was incubated with PP2A samples in dephosphorylation buffer (20 mM Tris 7.5, 1 mM DTT) at 30° C. for 30 minutes. The reaction was stopped by adding SDS loading buffer and the samples were loaded onto SDS-PAGE. The phosphorylation status of Tau was examined by western blot using an antibody (Biosource) that specifically recognizes phosphorylated Ser396 of Tau. Antibody recognizing both the phosphorylated and non-phosphorylated Tau (Invitrogen) was used as control.

### Example 3

### Crystallographic Data and Refinement for PP2A Crystal Structure

The following data was collected and characterized from the crystal of PP2A as described herein. The atomic coordinates of the crystal of PP2A as described herein have also been deposited in the Protein Data Bank with the accession code 3DW8, which is hereby incorporated in its entirety. The data is also shown in Appendix I. Statistics regarding the crystals are presented in Table 1.

TABLE 1
Crystallographic data and refinement.

| Protein | PP2A-Aα-Bα-Cα |
|---|---|
| Beamline/wavelength | NSLS-X29/1.0809 Å |
| Space group | C2 |
| Resolution (outer shell) (Å) | 50.0-2.85 (2.95-2.85) |
| Total observations | 275,520 |
| Unique observations | 87,353 |
| Redundancy (outer shell) | 3.2 (3.2) |
| Data coverage (outer shell) | 98.9% (99.9%) |
| R_{sym} (outer shell) | 0.050 (0.465) |
| Refinement: | |
| Resolution range (Å) | 50.0-2.85 Å |
| Number of reflections (\|F\| > 0) | 83,615 |
| Data coverage | 95.3% |
| R_{working}/R_{free} | 0.228/0.286 |
| Number of atoms | 20,708 |
| Number of waters | 0 |
| R.m.s.d. bond length (Å) | 0.0096 |
| R.m.s.d. bond angles (degree) | 1.47 |
| Ramachandran Plot: | |
| Most favored (%) | 81.8 |
| Additionally allowed (%) | 17.1 |
| Generously allowed (%) | 0.8 |
| Disallowed (%) | 0.3 |

Rsym = ΣₕΣᵢ | I_{h, i} − Iₕ |/ΣₕΣᵢ I_{h, i}, where Iₕ is the mean intensity of the i observations of symmetry related reflections of h. R = Σ | F_{obs} − F_{calc} |/ΣF_{obs}, where F_{obs} = F_{P}, and F_{calc} is the calculated protein structure factor from the atomic model (R_{free} was calculated with 5% of the reflections). R.m.s.d. in bond lengths and angles are the deviations from ideal values, and the r.m.s.d. deviation in B factors is calculated between bonded atoms.

1. A composition comprising a crystal of a PP2A holoenzyme, wherein said holoenzyme comprises an A subunit, a catalytic subunit (C), and a regulatory (B) subunit, wherein said regulatory subunit is Bα.

2. The composition of claim 1, wherein said A subunit comprises residues 1-589 of SEQ ID NO: 1; Bα comprises residues 1-447 of SEQ ID NO: 2; and the catalytic subunit comprises residues 1-309 of SEQ ID NO: 3.

3. The composition of claim 1, wherein said A subunit comprises residues 9-589 of SEQ ID NO: 1.

4. The composition of claim 1, wherein said B subunit comprises residues 8-446 of SEQ ID NO: 2.

5. The composition of claim 1, wherein said catalytic subunit comprises residues 6-293 of SEQ ID NO: 3.

6. The composition of claim 1, wherein said catalytic subunit is methylated.

7. The composition of claim 1 further comprising microcystin-LR (MCLR).

8. The composition of claim 1, wherein said crystal has space group of I4, P1 or C2.

9. The composition of claim 8, wherein said crystal in a space group of P1 has unit cell dimensions, ±2%, of a=124 Å b=141 Å, c=141 Å, α=79° β=64°, γ=64°.

10. The composition of claim 9, wherein said crystal comprises four complexes in each asymmetric unit.

11. The composition of claim 8, wherein said crystal in a space group of C2 has unit cell dimensions, ±2%, of a=247 Å b=121 Å, c=172 Å, α=90° β=133°, γ=90°.

12. The composition of claim 11, wherein said crystal comprises two complexes in each asymmetric unit.

13. The composition of claim 8, wherein said crystal in a space group of I4 has unit cell dimensions, ±2%, of a=182 Å b=182 Å, c=124 Å, α=90° β=90°, γ=90°.

14. The composition of claim 13, wherein said crystal comprises 1 complex in each asymmetric unit.

15. The composition of claim 1 wherein the crystal diffracts X-rays for a determination of structure coordinates to a resolution of a value equal to or less than about 5.0 angstroms.

16. The composition of claim 1 wherein the crystal diffracts X-rays for a determination of structure coordinates to a resolution of a value equal to or less than about 2.85 angstroms.

17. The composition of claim 1 wherein said crystal comprising a PP2A holoenzyme with a structure as defined by the coordinates as shown in Appendix 1.

18. The composition of claim 1, wherein a methionine is replaced with selenomethionine.

19. A method for preparing a PP2A holoenzyme modulating compound comprising:
applying a three-dimensional molecular modeling algorithm to the atomic coordinates of at least a portion of the PP2A holoenzyme;
determining spatial coordinates of the at least a portion of the PP2A holoenzyme;
electronically screening stored spatial coordinates of candidate compounds against the spatial coordinates of the at least a portion of the PP2A holoenzyme;
identifying a compound that is substantially similar to the at least a portion of the PP2A holoenzyme; and
synthesizing the identified compound,
wherein said PP2A holoenzyme comprises an A subunit, a catalytic subunit (C), and a regulatory (B) subunit, wherein said regulatory subunit is Bα.

20. The method of claim 19, further comprising identifying a candidate compound that deviates from the atomic coordinates of the at least a portion of the PP2A holoenzyme by a root mean square deviation of less than about 10 angstroms.

21. The method of claim 19, further comprising testing the identified compound for binding at least a portion of the PP2A holoenzyme.

22. The method of claim 19, further comprising testing the identified compound for inhibiting PP2A phosphatase activity.

23. The method of claim 19, wherein said PP2A dephosphorylation of Tau is measured.

24. The method of claim 19, further comprising testing the identified compound for inhibiting binding of PP2A to Tau.

25. The method of claim 19, further comprising testing the identified compound to determine if it inhibits or enhances tyrosine phosphorylation, serine phosphorylation, threonine phosphorylation or a combination thereof modulated by PP2A.

26. The method of claim 19, wherein the step of electronically screening stored spatial coordinates further comprises identifying a compound that has a shape, a charge distribution, a size or a combination thereof substantially similar to a portion of the PP2A holoenzyme.

27. The method of claim 19, wherein the at least a portion of the PP2A holoenzyme comprises the interface between the A subunit and the B subunit.

28. The method of claim 19, wherein the at least a portion of the PP2A holoenzyme comprises the a residue of PP2A that binds to Tau.

29. The method of claim 27 wherein the identified compound interrupts the interface between A subunit and the B subunit.

30. The method of claim 28 wherein the identified compound interrupts the interface between the B subunit and Tau.

31. The method of claim 19, wherein the identified compound binds to the B subunit.

32. A pharmaceutical composition comprising:
an effective amount of a compound having a three-dimensional structure corresponding to atomic coordinates of at least a portion of a PP2A holoenzyme, wherein said holoenzyme comprises an A subunit, a catalytic subunit (C), and a regulatory (B) subunit, wherein said regulatory subunit is Bα; and
a pharmaceutically acceptable excipient or carrier.

33. The pharmaceutical composition of claim 33, wherein the compound binds to the B subunit.

34. A system for identifying PP2A modulators comprising:
a processor; and
a processor readable storage medium in communication with the processor readable storage medium comprising the atomic coordinates of at least a portion of a PP2A holoenzyme, wherein said holoenzyme comprises an A subunit, a catalytic subunit (C), and a regulatory (B) subunit, wherein said regulatory subunit is Bα.

35. The system of claim 34, wherein the processor readable storage medium further comprises one or more programming instructions for:
applying a three-dimensional modeling algorithm to the atomic coordinates of the PP2A holoenzyme;
determining spatial coordinates of at least a portion of the PP2A holoenzyme;
electronically screening spatial coordinates of candidate compounds with the spatial coordinates of the at least a portion of the PP2A holoenzyme; and
identifying a candidate compound whose spatial coordinates are substantially similar to the spatial coordinates of the at least a portion of the PP2A holoenzyme; or
identifying a candidate compound whose spatial coordinates are substantially complementary to the spatial coordinates of the at least a portion of the PP2A holoenzyme.

36. The system of claim 35, wherein the one or more programming instructions for identifying a candidate compound whose spatial coordinates are substantially similar to the spatial coordinates of the at least a portion of the PP2A holoenzyme comprise one or more programming instructions for identifying a compound that deviates from the spatial coordinates of the at least a portion of the PP2A holoenzyme by a user defined threshold.

37. The system of claim 35, wherein the one or more programming instructions for identifying a compound whose spatial coordinates are substantially similar to the at least a portion of the PP2A holoenzyme comprise one or more programming instructions for identifying a compound having one or more of:
a size within a user defined threshold;
a charge within a user defined threshold; or
a shape with a user defined threshold.

38. The system of claim 35, wherein the one or more programming instructions for electronically screening spatial coordinates of a candidate compound comprises one or more programming instructions for simulating binding of the candidate compound to the PP2A holoenzyme.

39. The system of claim 34, further comprising an output device in communication with the processor.

40. The system of claim 34, wherein the processor readable storage medium further comprises one or more programming instructions for:
applying a three-dimensional modeling algorithm to the atomic coordinates of PP2A holoenzyme;
determining spatial coordinates of at least a portion of the PP2A holoenzyme;
generating a visual signal and relaying the visual signal to the output device; and
electronically designing a compound that is substantially similar to the at least a portion of the PP2A holoenzyme; or
electronically designing a compound that is substantially complementary to the at least a portion of the PP2A holoenzyme.

41. A PP2A holoenzyme binding compound comprising a molecule having a three-dimensional structure corresponding to atomic coordinates derived from at least a portion of an atomic model of the PP2A holoenzyme, wherein said holoenzyme comprises an A subunit, a catalytic subunit (C), and a regulatory (B) subunit, wherein said regulatory subunit is Bα.

42. The compound of claim 41, wherein the molecule is an inhibitor of PP2A.

43. The compound of claim 41, wherein the molecule inhibits the interaction between the PP2A holoenzyme and Tau.

44. The compound of claim 41, wherein the molecule has a three-dimensional structure corresponding to atomic coordinates of at least a portion subunit B of PP2A bound to subunit A,
wherein the compound makes interactions with the B subunit of protein phosphatase 2A (PP2A) holoenzyme that correspond to at least a portion of the interactions observed between the B subunit of protein phosphatase 2A (PP2A) holoenzyme and the A subunit of PP2A.

45. The compound of claim 41, wherein the molecule has a three-dimensional structure corresponding to atomic coordinates of at least a portion subunit B of PP2A that binds to Tau,
wherein the compound makes interactions with the B subunit of protein phosphatase 2A (PP2A) holoenzyme that correspond to at least a portion of the residues that interact with Tau.

46. The compound of claim 44, wherein the molecule binds the B subunit of protein phosphatase 2A (PP2A) at a binding site for the A subunit of PP2A.

47. The compound of claim 45, wherein the molecule binds the B subunit of protein phosphatase 2A (PP2A) at a binding site for Tau.

48. The compound of claim 41, wherein the molecule is substantially complementary to a portion of PP2A.

49. The compound of claim 41, wherein the molecule is substantially complementary to a portion of the B subunit of the protein phosphatase 2A (PP2A) holoenzyme.

50. The compound of claim 41, wherein the molecule binds to at least a portion of the B subunit of PP2A with a greater affinity than a naturally occurring substrate.

51. The compound of claim 41, wherein the molecule inhibits or enhances protein phosphatase 2A (PP2A) catalyzed activity.

52. The compound of claim 41, further comprising a pharmaceutically acceptable excipient or carrier.

53. The compound of claim 41, wherein the molecule deviates from the atomic coordinates of the at least a portion of the PP2A holoenzyme by a root mean square deviation of less than about 10 angstroms.

54. The compound of claim 41, wherein the molecule deviates from the atomic coordinates of the at least a portion of the PP2A holoenzyme by a root mean square deviation of less than about 2 angstroms.

55. A recombinant polypeptide comprising a PP2A binding fragment of Tau.

56. The polypeptide of claim 57, wherein said PP2A binding fragment of Tau comprises residues 197-259 and/or residues 265-328 of SEQ ID NO: 4.

57. An isolated nucleic acid encoding a polypeptide comprising a PP2A binding fragment of Tau.

58. The nucleic acid of claim 59, wherein said nucleic acid encodes a polypeptide comprising residues 197-259 and/or residues 265-328 of SEQ ID NO: 4.


SEQUENCE LISTING (4 sequences)

SEQ ID NO: 1 - protein, 589 aa, homo sapiens
MAAADGDDSLYPIAVLIDELRNEDVQLRLNSIKKLSTIALALGVERTRSELLPFLTDTIYDEDEVLLALAEQLGTFTTLVGGPEYVHCLLPPLESLATVEETVVRDKAVESLRAISHEHSPSDLEAHFVPLVKRLAGGDWFTSRTSACGLFSVCYPRVSSAVKAELRQYFRNLCSDDTPMVRRAAASKLGEFAKVLELDNVKSEIIPMFSNLASDEQDSVRLLAVEACVNIAQLLPQEDLEALVMPTLRQAAEDKSWRVRYMVADKFTELQKAVGPEITKTDLVPAFQNLMKDCEAEVRAAASHKVKEFCENLSADCRENVIMSQILPCIKELVSDANQHVKSALASVIMGLSPILGKDNTIEHLLPLFLAQLKDECPEVRLNIISNLDCVNEVIGIRQLSQSLLPAIVELAEDAKWRVRLAIIEYMPLLAGQLGVEFFDEKLNSLCMAWLVDHVYAIREAATSNLKKLVEKFGKEWAHATIIPKVLAMSGDPNYLHRMTTLFCINVLSEVCGQDITTKHMLPTVLRMAGDPVANVRFNVAKSLQKIGPILDNSTLQSEVKPILEKLTQDQDVDVKYFAQEALTVLSLA

SEQ ID NO: 2 - protein, 447 aa, Rattus norvegicus
MAGAGGGNDIQWCFSQVKGAVDDDVAEADIISTVEFNHSGELLATGDKGGRVVIFQQEQENKIQSHSRGEYNVYSTFQSHEPEFDYLKSLEIEEKINKIRWLPQKNAAQFLLSTNDKTIKLWKISERDKRPEGYNLKEEDGRYRDPTTVTTLRVPVFRPMDLMVEASPRRIFANAHTYHINSISINSDYETYLSADDLRINLWHLEITDRSFNIVDIKPANMEELTEVITAAEFHPNSCNTFVYSSSKGTIRLCDMRASALCDRHSKLFEEPEDPSNRSFFSEIISSISDVKFSHSGRYMMTRDYLSVKVWDLNMENRPVETYQVHEYLRSKLCSLYENDCIFDKFECCWNGSDSVVMTGSYNNFFRMFDRNTKRDITLEASRENNKPRTVLKPRKVCASGKRKKDEISVDSLDFNKKILHTAWHPKENIIAVATTNNLYIFQDKVN

SEQ ID NO: 3 - protein, 309 aa, homo sapiens
MDEKVFTKELDQWIEQLNECKQLSESQVKSLCEKAKEILTKESNVQEVRCPVTVCGDVHGQFHDLMELFRIGGKSPDTNYLFMGDYVDRGYYSVETVTLLVALKVRYRERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL

SEQ ID NO: 4 - protein, 383 aa, homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL